(19) United States
(12) Patent Application Publication
(10) Pub. No.: US 2025/0091990 A1
(43) Pub. Date: Mar. 20, 2025

(54) DEUTERATED ORGANIC COMPOUNDS AND USES THEREOF

(71) Applicant: ENGRAIL THERAPEUTICS, INC., San Diego, CA (US)

(72) Inventors: Krishna VADODARIA, San Diego, CA (US); Kimberly VANOVER, Tulsa, OK (US); Jordi SERRATS, San Diego, CA (US); Vikram SUDARSAN, San Diego, CA (US); David GARVEY, Dover, MA (US)

(21) Appl. No.: 18/726,550

(22) PCT Filed: Jan. 3, 2023
(86) PCT No.: PCT/US2023/010050

§ 371 Date: Jul. 3, 2024

Related U.S. Application Data
(60) Provisional application No. 63/296,134, filed on Jan. 3, 2022.
(60) Provisional application No. 63/345,007, filed on May 23, 2022.
(60) Provisional application No. 63/384,992, filed on Nov. 25, 2022.

(51) Int. Cl. C07D 207/14; A61K 31/40; A61P 25/18; C07B 59/00

(52) U.S. Cl. CPC C07D 207/14; A61K 31/40; A61P 25/18; C07B 59/002

(57) ABSTRACT

Provided are compounds of Formula I, described herein, processes for their preparation, their use as pharmaceuticals, and pharmaceutical compositions comprising them and intermediates used in their preparation. Compounds of Formula I are useful, for instance, in modulating dopamine and serotonin neurotransmission and treating disorders that may benefit from the same, such as schizophrenia and depression.

This application claims priority to U.S. Provisional Application No. 63/296,134 filed Jan. 3, 2022, U.S. Provisional Application No. 63/345,007 filed May 23, 2022, and U.S. Provisional Application No. 63/384,992 filed Nov. 25, 2022, the contents of each of which are hereby incorporated by reference in their entireties.

### FIELD

Provided are compounds of Formula I, described below, processes for their preparation, their use as pharmaceuticals, and pharmaceutical compositions comprising them and intermediates used in their preparation. Compounds of Formula I are useful, for instance, in modulating dopamine and serotonin neurotransmission and treating disorders that may benefit from the same, such as schizophrenia and depression.

### BACKGROUND

Dopamine is involved in a variety of central nervous system functions, including voluntary movement, feeding, affect, reward, sleep, attention, working memory, and learning. Serotonin also is involved in a variety of central nervous system functions, including mood, cognition, reward, learning, memory, and various physiological processes. Accordingly, dopaminergic and/or serotonergic dysfunction can lead to diseases such as schizophrenia and depression.

When released from presynaptic terminals, dopamine activates members of a family of G protein-coupled dopamine receptors D1-D5. Dopamine receptors (D1-D5) are divided into two groups, the D1-like (D1 and D5) and the D2-like (D2, D3, and D4). Activation of D1-like receptors activates adenylyl cyclase and increases cAMP levels. D2-like receptors are inhibitory. Activation of D2-like receptors inhibits activation of adenylyl cyclase.

D1-like receptors are found postsynaptically on dopamine-receptive cells, while D2-like dopamine receptors are expressed both postsynaptically on dopamine target cells and presynaptically on dopaminergic neurons.

Fourteen serotonin receptor subtypes, grouped into sub-families, mediate effects of serotonin (5-HT). The 5-HT1A receptor subtype, a major receptor subtype, exists as presynaptic autoreceptor in serotonin neurons in the raphe nuclei and as postsynaptic heteroreceptors in the prefrontal cortex, hippocampus, septum, and hypothalamus. Signaling mechanisms of 5-HT1A receptors in the raphe nuclei may be different from 5-HT1A receptors in other brain regions. Activation of 5-HT1A postsynaptic receptors can elicit increased dopamine release. The 5-HT2A receptor subtype is enriched in cortex and is linked to phosphatidylinositol turnover and also modulates dopamine release. 5-HT2A receptor antagonists have antipsychotic properties, while 5-HT2A receptor agonism is thought to be associated with cognition-enhancing and hallucinogenic properties. The hallucinogenic effects of lysergic diethylamide (LSD) and psilocybin are thought to arise from their 5-HT2A receptor agonism. 5-HT2A agonism has also been reported to promote neural plasticity and reduce depression.

Antipsychotics are used to manage psychosis, in particular schizophrenia. A hallmark of antipsychotics is D2 receptor antagonism. D2 receptor antagonism is effective in reducing positive symptoms of schizophrenia (for instance, hallucinations and delusions), but often also produces extrapyramidal side effects, including parkinsonism, akathisia, and tardive dyskinesia, increases prolactin, and may exacerbate negative symptoms of schizophrenia (for instance, loss of interest and motivation in life and activities, social withdrawal, and anhedonia). A key feature of atypical antipsychotics is D2 receptor antagonism in combination with 5-HT2A receptor antagonism, which may explain their enhanced efficacy and reduced extrapyramidal motor side effects (EPS) compared to typical antipsychotics. Many psychotic patients also suffer from depression, which may be left untreated by current medications. However, some atypical antipsychotics are used adjunctively to serotonergic antidepressants to improve response in major depressive disorder.

Because imbalances in dopamine and serotonin can lead to a variety of disorders and current medications may not be able to effectively modulate levels of both, new compounds that can modulate dopamine and serotonin neurotransmission are needed, as are methods of treating diseases that involve imbalances in dopamine and serotonin.

### BRIEF SUMMARY

Provided is a compound of Formula I:

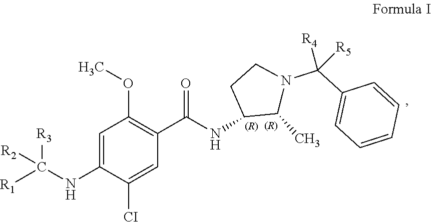

wherein:
R₁, R₂, R₃, R₄, and R₅ are independently selected from H and D; and
at least one of R₁, R₂, and R₃ is D;
in free or salt form.

Further provided are pharmaceutical compositions comprising compounds of Formula I, processes for preparing compounds of Formula I, and pharmaceutical uses of compounds of Formula I, for instance, as an anti-anhedonic agent and to treat schizophrenia and depression.

Further areas of applicability of the present invention will become apparent from the detailed description provided hereinafter. It should be understood that the detailed description and specific examples, while indicating preferred embodiments of the invention, are intended for purposes of illustration only and are not intended to limit the scope of the invention.

### BRIEF DESCRIPTION OF THE DRAWINGS

FIG. 1 shows disappearance of cis (R,R) nemonapride in human hepatocytes.

FIG. 2 shows disappearance of the compound of Example 1 (A2) in human hepatocytes.

FIG. 3 shows plasma concentration (ng/ml) in rats of cis (R,R) nemonapride and the compound of Example 1 (A2) following a single PO dose of 0.5 mg/kg.

FIG. 4 shows extended brain enrichment of the compound of Example 1 (A2) in rats following a single PO dose of 0.5 mg/kg compared to plasma levels.

FIG. 5 shows extended brain enrichment of the compound of Example 1 (A2) in rats following a single PO dose of 5 mg/kg compared to plasma levels.

FIG. 6 shows average brain concentration (ng/ml) in rats of cis (R,R) nemonapride and the compound of Example 1 (A2) when administered at a single PO dose of 0.5 mg/kg.

FIG. 7 shows average plasma concentration (ng/ml) in rats of cis (R,R) nemonapride and the compound of Example 1 (A2) following single oral administration of 2.5 mg/kg.

FIG. 8 shows extended brain enrichment of the compound of Example 1 (A2) in rats following a single oral administration of 2.5 mg/kg compared to plasma levels.

FIG. 9 shows average brain concentration (ng/ml) of cis (R,R) nemonapride and the compound of Example 1 (A2) following a single oral administration of 2.5 mg/kg to rats.

FIG. 10 shows D2 receptor occupancy of cis (R,R) nemonapride and the compound of Example 1 (A2) when administered orally at a dose of 2.5 mg/kg to rats.

FIG. 11 shows average plasma and brain concentrations (ng/ml) of cis (R,R) nemonapride following a single oral administration of 2.5 mg/kg to rats.

FIG. 12A shows response bias in probabilistic reward task for the compound of Example 1 (A2) when administered at doses of 0.5, 1, and 2.5 mg/kg to rats.

FIG. 12B shows discriminability in the probabilistic reward task for the compound of Example 1 (A2) when administered at doses of 0.5, 1, and 2.5 mg/kg to rats.

FIG. 13 shows a pharmacokinetic:pharmacodynamic model for the compound of Example 1 (A2) for an oral 1 mg/kg oral dose.

### DETAILED DESCRIPTION

The following description of the preferred embodiment(s) is merely exemplary in nature and is in no way intended to limit the invention, its application, or uses.

In the event of a conflict in a definition in the present disclosure and that of a cited reference, the present disclosure controls.

D2- and D3-receptors are expressed both postsynaptically on dopamine target cells and presynaptically on dopamine neurons. Dopamine receptors are mainly located on non-dopamine neurons. Dopamine receptors on dopamine neurons are called autoreceptors. Autoreceptors contribute to regulating dopamine neuron activity and controlling the synthesis, release, and uptake of dopamine.

Presynaptic D2-like dopamine autoreceptors regulate dopamine release. A low dose of a D2-like receptor antagonist may preferentially block presynaptic autoreceptors and increase dopamine release, while a high dose may block postsynaptic receptors and decrease dopamine neurotransmission. Relatively high occupancy of D2-like receptors has been associated with antipsychotic effects, while lower occupancy has been associated with antidepressant effects.

Anhedonia is a core symptom of major depressive disorder (MDD) and is associated with inadequate response to approved selective serotonin reuptake inhibitors (SSRIs) and serotonin norepinephrine reuptake inhibitors (SNRIs) and psychotherapy (e.g., cognitive behavioral therapy (CBT)) and neurostimulation (e.g., transcranial magnetic stimulation (TMS)). There remains a need for effective treatment of MDD characterized by anhedonia. Despite a range of available therapies, up to 50% of people suffering from MDD fail to respond to treatments, and only about 30% of patients fully recover after receiving currently available antidepressants and treatment outcomes are even poorer for MDD individuals with anhedonia.

Depletion of dopamine/catecholamines induces symptoms of depression and anhedonia. Increasing dopamine neurotransmission can alleviate symptoms of depression and anhedonia. However, while a high dose of a dopamine D2/D3 agonist may activate dopamine post-synaptic receptors, it can also be poorly tolerated (e.g., nausea/vomiting). Low dose of a dopamine D2/D3 receptor antagonist may preferentially block pre-synaptic dopamine autoreceptors and increase dopamine release without being poorly tolerated.

Besides MDD, anhedonia also plays a role in bipolar disorder, schizophrenia, post-traumatic stress disorder, and substance use disorder. Despite its role in many disorders, there are no approved medications to treat anhedonia. Decreased serotonergic activity has been implicated in anxiety and depression. Increasing serotonin neurotransmission may alleviate symptoms of anxiety and depression and be helpful for anxious depression.

The IUPAC name of nemonapride is (±)-cis-N-(1-Benzyl-2-methylpyrrolidin-3-yl)-5-chloro-2-methoxy-4-methylaminobenzamide. Nemonapride is described in U.S. Pat. No. 4,210,660 as a strong central nervous system depressant, in particular a strong antipsychotic.

Nemonapride is a dopamine D2/D3/D4 receptor antagonist. Nemonapride is approved in Japan and South Korea for treatment of schizophrenia. Nemonapride is supplied as 3 mg and 10 mg tablets. The approved daily dose of nemonapride for schizophrenia is 9 to 36 mg given orally in divided doses after meals. The dose can be increased up to 60 mg daily.

The nemonapride prescribing information indicates that the elimination half-life when nemonapride 3 mg and 6 mg was administered orally to healthy adults was 2.3 to 4.5 hours. Urinary metabolites of nemonapride result from debenzylation and N-demethylation. See Emilace package insert.

In addition to being a dopamine D2/D3/D4 receptor antagonist, nemonapride is also a 5-HT1A agonist. Further, nemonapride has been reported to bind to 5-HT2A receptors, however, the inventors are not aware of any publication that reports its functional effect at that receptor. Yet, as an antipsychotic, it may be expected that nemonapride is a 5-HT2A receptor antagonist because a key feature of atypical antipsychotics is D2 receptor antagonism in combination with 5-HT2A receptor antagonism or inverse agonism.

When a drug is used as a mixture of stereoisomers, it is not possible to predict what properties (e.g., biological target, pharmacokinetics) each stereoisomer has, especially a drug that has multiple biological targets.

Compounds of Formula I disclosed herein are D2/D3/D4 receptor antagonists, 5-HT1A agonists, and 5-HT2A partial agonists. The deuterated compound of Example 1 shows higher 5-HT2A agonism than its non-deuterated analog (see Example 3). D2/D3/D4 receptor antagonism in combination with 5-HT1A and 5-HT2A agonism is a unique activity profile, which may allow for different modulation of dopamine and serotonin neurotransmission compared to other D2/D3/D4 receptor antagonists. Other substituted benzamides tested—R-remoxipride, S-remoxipride, R-sulpiride, R-sulfopride, and S-suflopride—do not even bind to the 5-HT2A receptor in vitro (labeled-Ketansrin competition assay).

As noted above, D2/D3/D4 receptor antagonism in combination with 5-HT1A and 5-HT2A agonism is a unique activity profile, which may allow for different modulation of dopamine and serotonin neurotransmission compared to other D2/D3/D4 receptor antagonists. For instance, D2/D3/D4 postsynaptic receptor antagonism reduces psychosis, particularly in schizophrenia, by reducing dopamine neurotransmission. High doses that target >60% receptor occupancy may be associated with D2 antagonist mediated side effects such as extrapyramidal motor side effects (EPS) and increased prolactin. However, 5-HT1A agonism may limit those high dose D2 antagonist related side effects, thus providing the compounds with a built-in safety feature when used at high dose as an antipsychotic. Partial 5-HT1A agonism also provides anxiolytic effects. Further, as partial 5-HT2A agonists, deuterated compounds disclosed herein may show enhanced antidepressant effects as seen with psychedelic antidepressants, for instance, rapid and long-lasting and with anxiolytic effects, yet at the same time hallucinogenic and fear/anxiety effects may not be as pronounced as with a full 5-HT2A agonist. And, D2 antagonism may also block 5-HT2A hallucinogenic effects.

Thus, as D2/D3 antagonists and 5-HT2A partial agonists, compounds of Formula I may provide psychedelic-like antidepressant efficacy at low doses (e.g., doses lower than those of nemonapride used to treat schizophrenia), but also have built-in protection against 5-HT2A mediated hallucinations and without fear/anxiety. Further, as D2/D3 antagonists and 5-HT1A agonists, compounds of Formula I may act as antipsychotics at high doses, but have built-in protection against high dose D2 antagonist related side effects.

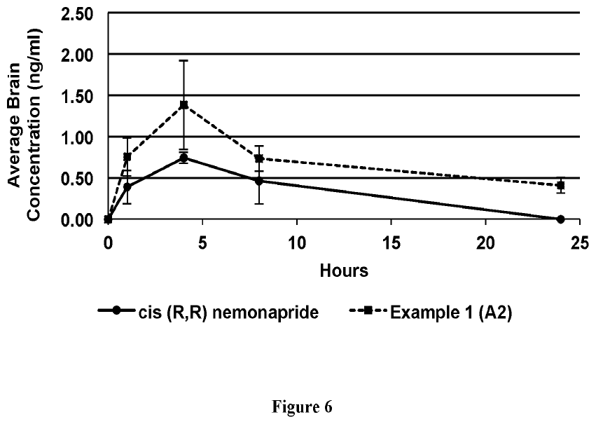

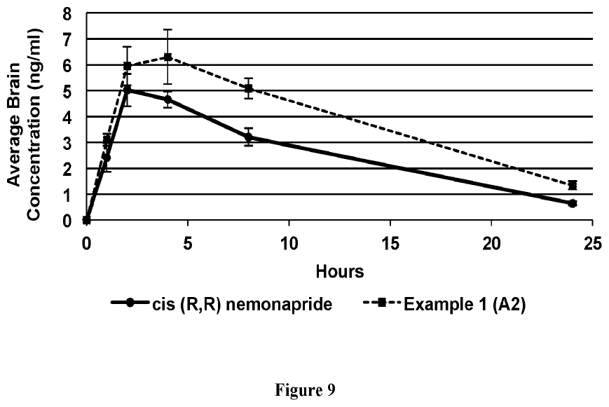

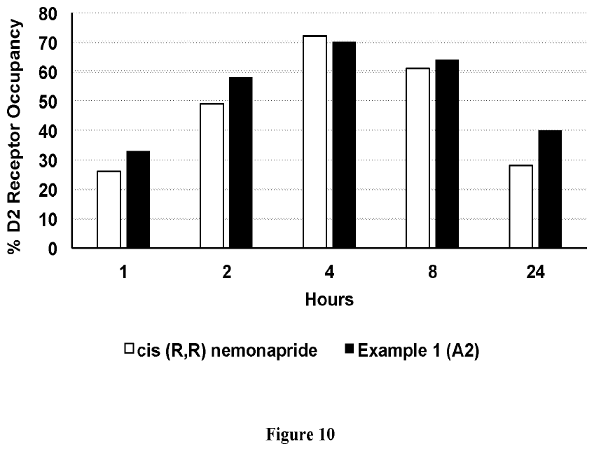

Pharmacokinetics of deuterated compounds disclosed herein are beneficial. Plasma pharmacokinetics of N-[(2R,3R)-1-benzyl-2-methylpyrrolidin-3-yl]-5-chloro-2-methoxy-4-(methylamino)benzamide (cis (R,R) nemonapride) and the deuterated compound of Example 1 (A2) are similar (see Example 5). However, despite similar plasma pharmacokinetics, Examples 5 and 6 show that a compound of Formula I (the deuterated compound of Example 1) has enriched and retained brain levels compared to its non-deuterated analog and higher receptor occupancy levels at 1, 2, 8, and 24 hours. For instance, FIGS. 6 and 9 show that at 8 hours, brain levels of the compound of Example 1 (A2) are similar to the highest levels of cis (R,R) nemonapride measured, which occur at shorter time. The deuterated compound of Example 1 also shows extended brain enrichment compared to plasma levels of the compound. The brain:plasma exposure supports once-daily dosing. Enriched brain levels, higher receptor occupancy levels, and extended brain enrichment compared to plasma levels are beneficial features that allows for higher and more sustained receptor occupancy with less frequent dosing and may be associated with fewer peripheral side effects. Further, the receptor occupancy curve of the deuterated compound of Example 1 (see FIG. 10) exhibits more moderate changes between peaks and troughs (a flatter curve) compared to its non-deuterated analog, which should provide more consistent and stable levels of dopamine and serotonin neurotransmission. Receptor occupancy levels can be maintained in a desired range with a convenient dosing regime. In contrast, nemonapride is taken in multiple doses per day.

Compounds that are D2/D3/D4 receptor antagonists, 5-HT1A receptor agonists, and 5-HT2A receptor partial agonists modulate dopamine and serotonin neurotransmission and are therefore useful in treating disorders involving dopamine and serotonin signaling pathways, for instance, disorders involving D2, D3, D4, 5-HT1A, and/or 5-HT2A receptors.

Provided is a compound of Formula I:

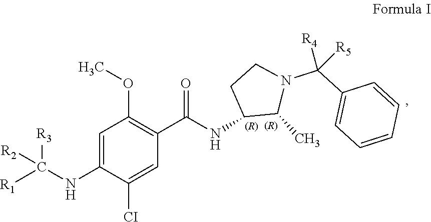

wherein:
R₁, R₂, R₃, R₄, and R₅ are independently selected from H and D; and
at least one of R₁, R₂, and R₃ is D;
in free or salt form.

Further provided are compounds of Formula I as follows:
1.1 Formula I, wherein the compound is in pharmaceutically acceptable salt form.
1.2 Formula I, wherein the compound is in free form.
1.3 Any of Formula I, 1.1, or 1.2, wherein R₁, R₂, and R₃ are D.
1.4 Any of Formula I or 1.1-1.3, wherein R₄ and R₅ are D.
1.5 Any of Formula I or 1.1-1.4, wherein each of R₁, R₂, R₃, R₄, and R₅ are D.
1.6 Any of Formula I or 1.1-1.5, wherein the compound is:

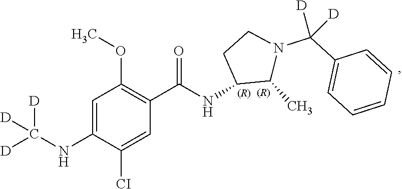

in free or salt form, e.g., in free or pharmaceutically acceptable salt form, e.g., in free form.
1.7 Any of Formula I or 1.1-1.6, wherein the designation of deuterium (i.e., D) at a position means that position has a significantly greater than natural abundance of deuterium at that position (e.g., greater than 0.1%, or greater than 0.5%, or greater than 1%, or greater than 5%). Any atom not designated as a particular isotope is present at natural isotopic abundance.
1.8 Any of Formula I or 1.1-1.7, wherein the compound, in free or salt form (e.g., pharmaceutically acceptable salt form), has greater than 50% incorporation of deuterium (i.e., D) at one or more positions (e.g., at all positions) designated as deuterium (i.e., D), e.g., greater than 60%, or greater than 70%, or greater than 80%, or greater than 90%, or greater than 95%, or greater than 96%, or greater than 97%, or greater than 98%, or greater than 99%. For instance, any of Formula I or 1.1-1.7, wherein the compound, in free or salt form (e.g., pharmaceutically acceptable salt form), has greater than 50% incorporation of deuterium (i.e., D) at each position designated as deuterium (i.e., D), e.g., greater than 60%, or greater than 70%, or greater than 80%, or greater than 90%, or greater than 95%, or greater than 96%, or greater than 97%, or greater than 98%, or greater than 99%.
1.9 Any of Formula I or 1.1-1.8, wherein the compound is substantially stereoisomerically pure. For instance, wherein the compound has a stereoisomeric excess of greater than 90%, e.g., a stereoisomeric excess equal to or greater than 95%, e.g., a stereoisomeric excess equal to or greater than 96%, e.g., a stereoisomeric excess equal to or greater than 97%, e.g., a stereoisomeric excess equal to or greater than 98%, e.g., a stereoisomeric excess equal to or greater than 99%. For instance, wherein the compound is substantially diastereomerically and/or enantiomerically pure, e.g., wherein the compound is substantially diastereomerically and enantiomerically pure.
1.10 Any of Formula I or 1.1-1.9, wherein the compound is substantially diastereomerically pure. For instance, wherein the compound has a diastereomeric excess of greater than 90%, e.g., a diastereomeric excess equal to or greater than 95%, e.g., a diastereomeric excess equal to or greater than 96%, e.g., a diastereomeric excess equal to or greater than 97%, e.g., a diastereomeric excess equal to or greater than 98%, e.g., a diastereomeric excess equal to or greater than 99%.
1.11 Any of Formula I or 1.1-1.10, wherein the compound is substantially enantiomerically pure. For instance, wherein the compound has an enantiomeric excess of greater than 90%, e.g., an enantiomeric excess equal to or greater than 95%, e.g., an enantiomeric excess equal to or greater than 96%, e.g., an enantiomeric excess equal to or greater than 97%, e.g., an enantiomeric excess equal to or greater than 98%, e.g., an enantiomeric excess equal to or greater than 99%.
1.12 Any of Formula I or 1.1-1.11, wherein the compound has the stereochemical configuration as shown in Formula I.
1.13 Any of Formula I or 1.1-1.12, wherein the compound is in a pharmaceutical composition with a pharmaceutically acceptable carrier. For instance, any of Formula I or 1.1-1.12, wherein an effective amount of the compound is in a pharmaceutical composition with a pharmaceutically acceptable carrier.

Further provided is a pharmaceutical composition (Composition 1) comprising a compound of Formula I (e.g., any of Formula 1.1-1.13):

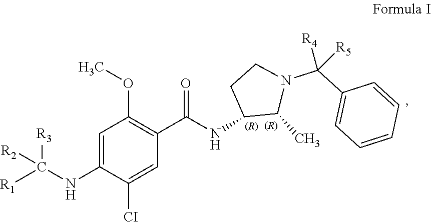

wherein:
R₁, R₂, R₃, R₄, and R₅ are independently selected from H and D; and
at least one of R₁, R₂, and R₃ is D;
in free or pharmaceutically acceptable salt form.

Further provided is Composition 1 as follows:
1.1 Composition 1, wherein the composition comprises a pharmaceutically acceptable carrier.
1.2 Composition 1 or 1.1, wherein the composition comprises the compound, in free or pharmaceutically acceptable salt form, as described in any of Formula I or 1.1-1.13 vide supra.
1.3 Any of Composition 1, 1.1, or 1.2, wherein the compound is in free form.
1.4 Any of Composition 1 or 1.1-1.3, wherein the compound of Formula I is:

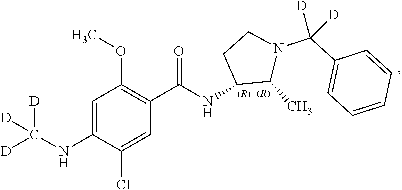

in free or pharmaceutically acceptable salt form, e.g., in free form.
1.5 Any of Composition 1 or 1.1-1.4, wherein the designation of deuterium (i.e., D) at a position means that position has a significantly greater than natural abundance of deuterium at that position (e.g., greater than 0.1%, or greater than 0.5%, or greater than 1%, or greater than 5%). Any atom not designated as a particular isotope is present at natural isotopic abundance.
1.6 Any of Composition 1 or 1.1-1.5, wherein the compound of Formula I, in free or pharmaceutically acceptable salt form, has greater than 50% incorporation of deuterium (i.e., D) at one or more positions (e.g., at all positions) designated as deuterium (i.e., D), e.g., greater than 60%, or greater than 70%, or greater than 80%, or greater than 90%, or greater than 95%, or greater than 96%, or greater than 97%, or greater than 98%, or greater than 99%. For instance, any of Composition 1 or 1.1-1.5, wherein the compound of Formula I, in free or pharmaceutically acceptable salt form, has greater than 50% incorporation of deuterium (i.e., D) at each position designated as deuterium (i.e., D), e.g., greater than 60%, or greater than 70%, or greater than 80%, or greater than 90%, or greater than 95%, or greater than 96%, or greater than 97%, or greater than 98%, or greater than 99%.
1.7 Any of Composition 1 or 1.1-1.6, wherein the composition is in oral or parenteral dosage form, e.g., oral dosage form, for instance, a tablet, capsule, solution, or suspension, for instance, a capsule or tablet.
1.8 Any of Composition 1 or 1.1-1.7, wherein the composition comprises a therapeutically effective amount of the compound of Formula I, in free or pharmaceutically acceptable salt form, e.g., a therapeutically effective amount of the compound of Formula I, in free or pharmaceutically acceptable salt form, for the prophylaxis or treatment of a disorder disclosed herein, e.g., a therapeutically effective amount of the compound of Formula I, in free or pharmaceutically acceptable salt form, for use in any of the methods disclosed herein.
1.9 Any of Composition 1 or 1.1-1.8, wherein the composition is substantially free of any other stereoisomeric form of Formula I. For instance, any of Composition 1 or 1.1-1.8, wherein the composition is substantially free of any other diastereomeric and/or enantiomeric form of Formula I, e.g., wherein the composition is substantially free of any other diastereomeric and enantiomeric form of Formula I.
1.10 Any of Composition 1 or 1.1-1.9, wherein the composition comprises less than 10% w/w (weight/weight) of any other stereoisomeric form of Formula I, e.g., less than 5% w/w of any other stereoisomeric form of Formula I, e.g., less than 4% w/w of any other stereoisomeric form of Formula I, e.g., less than 3% w/w of any other stereoisomeric form of Formula I, e.g., less than 2% w/w of any other stereoisomeric form of Formula I, e.g., less than 1% w/w of any other stereoisomeric form of Formula I.
1.11 Any of Composition 1 or 1.1-1.10, wherein the composition comprises less than 10% w/w of any other diastereomeric form of Formula I, e.g., less than 5% w/w of any other diastereomeric form of Formula I, e.g., less than 4% w/w of any other diastereomeric form of Formula I, e.g., less than 3% w/w of any other diastereomeric form of Formula I, e.g., less than 2% w/w of any other diastereomeric form of Formula I, e.g., less than 1% w/w of any other diastereomeric form of Formula I.
1.12 Any of Composition 1 or 1.1-1.11, wherein the composition comprises less than 10% w/w of any other enantiomeric form of Formula I, e.g., less than 5% w/w of any other enantiomeric form of Formula I, e.g., less than 4% w/w of any other enantiomeric form of Formula I, e.g., less than 3% w/w of any other enantiomeric form of Formula I, e.g., less than 2% w/w of any other enantiomeric form of Formula I, e.g., less than 1% w/w of any other enantiomeric form of Formula I.
1.13 Any of Composition 1 or 1.1-1.12, wherein the compound has the stereochemical configuration as shown in Formula I.
1.14 Any of Composition 1 or 1.1-1.13, wherein the composition comprises 1-60 mg of the compound of Formula I. For instance, any of Composition 1 or 1.1-1.13, wherein the composition comprises 1-10 mg, e.g., 1-9 mg (e.g., 1-8 mg) of the compound of Formula I. For instance, any of Composition 1 or 1.1-1.13, wherein the composition comprises 3 mg or 10 mg of the compound of Formula I. For instance, any of Composition 1 or 1.1-1.13, wherein the composition comprises 1 mg to less than 3 mg (e.g., 2 mg) of the compound of Formula I.
1.15 Any of Composition 1 or 1.1-1.14, wherein the composition is for once, twice, or three times daily dosing. For instance, any of Composition 1 or 1.1-1.14, wherein the composition is for once daily dosing.

Further provided are methods of prophylaxis or treatment of a central nervous system disorder (e.g., a brain disorder), for instance, a central nervous system disorder (e.g., a brain disorder) that benefits from modulating dopamine and/or serotonin transmission, in a patient in need thereof, wherein the method comprises administering to the patient a compound of Formula I, in free or pharmaceutically acceptable salt form (e.g., any of Formula I or 1.1-1.13 vide supra), or a pharmaceutical composition comprising a compound of Formula I, in free or pharmaceutically acceptable salt form (e.g., Formula 1.13 or any of Composition 1 or 1.1-1.15 vide supra), or a compound of Formula Ia or Compound A, in free or pharmaceutically acceptable salt form (vide infra), or a pharmaceutical composition comprising a compound of Formula Ia or Compound A, in free or pharmaceutically acceptable salt form (vide infra). Further provided are methods of prophylaxis or treatment of a central nervous system disorder (e.g., a brain disorder) that benefits from D2 receptor antagonism, D3 receptor antagonism, D4 receptor antagonism, 5-HT1A receptor agonism (e.g., 5-HT1A receptor partial agonism), and/or 5-HT2A receptor agonism (e.g., 5-HT2A receptor partial agonism) in a patient in need thereof, wherein the method comprises administering to the patient a compound of Formula I, in free or pharmaceutically acceptable salt form (e.g., any of Formula I or 1.1-1.13 vide supra), or a pharmaceutical composition comprising a compound of Formula I, in free or pharmaceutically acceptable salt form (e.g., Formula 1.13 or any of Composition 1 or 1.1-1.15 vide supra), or a compound of Formula Ia or Compound A, in free or pharmaceutically acceptable salt form (vide infra), or a pharmaceutical composition comprising a compound of Formula Ia or Compound A, in free or pharmaceutically acceptable salt form (vide infra). For instance, provided are methods as described below.

Provided is a method (Method 1) for treatment or prophylaxis of a disorder (e.g., a brain disorder) in a patient in need thereof, wherein the method comprises administering to the patient an effective amount of a compound of Formula Ia:

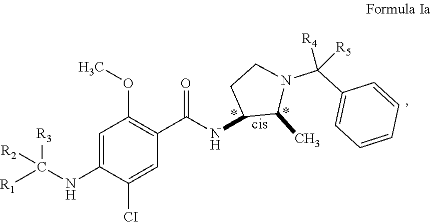

wherein:
the compound of Formula Ia has cis stereochemistry at the two stereocenters marked with asterisks in the drawing,
R₁, R₂, R₃, R₄, and R₅ are independently selected from H and D;
in free or pharmaceutically acceptable salt form.

Further provided is Method 1 as follows:
1.1 Method 1, wherein the method comprises administering (±)-cis-N-(1-Benzyl-2-methylpyrrolidin-3-yl)-5-chloro-2-methoxy-4-methylaminobenzamide (i.e. nemonapride), in free or pharmaceutically acceptable salt form, wherein (±)-cis-N-(1-Benzyl-2-methylpyrrolidin-3-yl)-5-chloro-2-methoxy-4-methylaminobenzamide does not show optical rotation in chloroform. For instance, wherein the method comprises administering (±)-cis-N-(1-Benzyl-2-methylpyrrolidin-3-yl)-5-chloro-2-methoxy-4-methylaminobenzamide (i.e. nemonapride), in free form, wherein (±)-cis-N-(1-Benzyl-2-methylpyrrolidin-3-yl)-5-chloro-2-methoxy-4-methylaminobenzamide does not show optical rotation in chloroform.
1.2 Method 1, wherein the method comprises administering an effective amount of Compound A:

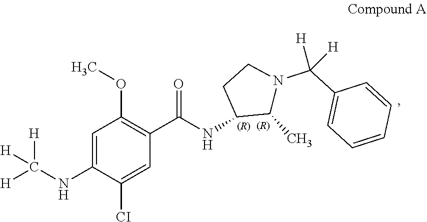

in free or pharmaceutically acceptable salt form. For instance, wherein the effective amount of Compound A, in free or pharmaceutically acceptable salt form, has a stereoisomeric excess of greater than 90%, e.g., a stereoisomeric excess equal to or greater than 95%, e.g., a stereoisomeric excess equal to or greater than 96%, e.g., a stereoisomeric excess equal to or greater than 97%, e.g., a stereoisomeric excess equal to or greater than 98%, e.g., a stereoisomeric excess equal to or greater than 99%. For instance, wherein the effective amount of Compound A, in free or pharmaceutically acceptable salt form, is substantially diastereomerically and/or enantiomerically pure, e.g., wherein the effective amount of Compound A, in free or pharmaceutically acceptable salt form, is substantially diastereomerically and enantiomerically pure. For instance, wherein the effective amount of compound A, in free or pharmaceutically acceptable salt form, has a diastereomeric and/or enantiomeric excess of greater than 90%, e.g., a diastereomeric and/or enantiomeric excess equal to or greater than 95%, e.g., a diastereomeric and/or enantiomeric excess equal to or greater than 96%, e.g., a diastereomeric and/or enantiomeric excess equal to or greater than 97%, e.g., a diastereomeric and/or enantiomeric excess equal to or greater than 98%, e.g., a diastereomeric and/or enantiomeric excess equal to or greater than 99%. For instance, wherein the effective amount of compound A, in free or pharmaceutically acceptable salt form, has a diastereomeric and enantiomeric excess of greater than 90%, e.g., a diastereomeric and enantiomeric excess equal to or greater than 95%, e.g., a diastereomeric and enantiomeric excess equal to or greater than 96%, e.g., a diastereomeric and enantiomeric excess equal to or greater than 97%, e.g., a diastereomeric and enantiomeric excess equal to or greater than 98%, e.g., a diastereomeric and enantiomeric excess equal to or greater than 99%.
1.3 Method 1, wherein the method comprises administering a compound of Formula I:

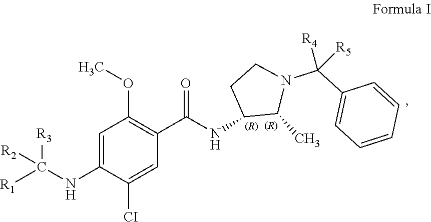

wherein:
R₁, R₂, R₃, R₄, and R₅ are independently selected from H and D; and
at least one of R₁, R₂, and R₃ is D;
in free or pharmaceutically acceptable salt form.
1.4 Method 1.3, wherein R₁, R₂, R₃, R₄, and R₅ are D.
1.5 Method 1.3 or 1.4, wherein the method comprises administering to the patient a compound of Formula I, in free or pharmaceutically acceptable salt form, as described in any of Formula I or 1.1-1.13 vide supra. For instance, Method 1.3 or 1.4, wherein the method comprises administering to the patient a pharmaceutical composition comprising a compound of Formula I, in free or pharmaceutically acceptable salt form, as described in any of Composition I or 1.1-1.15 vide supra.
1.6 Any of Method 1.3-1.5, wherein the effective amount of the compound of Formula I, in free or pharmaceutically acceptable salt form, has a stereoisomeric excess of greater than 90%, e.g., a stereoisomeric excess equal to or greater than 95%, e.g., a stereoisomeric excess equal to or greater than 96%, e.g., a stereoisomeric excess equal to or greater than 97%, e.g., a stereoisomeric excess equal to or greater than 98%, e.g., a stereoisomeric excess equal to or greater than 99%. For instance, wherein the effective amount of the compound of Formula I, in free or pharmaceutically acceptable salt form, is substantially diastereomerically and/or enantiomerically pure, e.g., wherein the effective amount of the compound of Formula I, in free or pharmaceutically acceptable salt form, is substantially diastereomerically and enantiomerically pure. For instance, wherein the effective amount of the compound of Formula I, in free or pharmaceutically acceptable salt form, has a diastereomeric and/or enantiomeric excess of greater than 90%, e.g., a diastereomeric and/or enantiomeric excess equal to or greater than 95%, a diastereomeric and/or enantiomeric excess equal to or greater than 96%, a diastereomeric and/or enantiomeric excess equal to or greater than 97%, e.g., a diastereomeric and/or enantiomeric excess equal to or greater than 98%, e.g., a diastereomeric and/or enantiomeric excess equal to or greater than 99%. For instance, wherein the effective amount of the compound of Formula I, in free or pharmaceutically acceptable salt form, has a diastereomeric and enantiomeric excess of greater than 90%, e.g., a diastereomeric and enantiomeric excess equal to or greater than 95%, e.g., a diastereomeric and enantiomeric excess equal to or greater than 96%, e.g., a diastereomeric and enantiomeric excess equal to or greater than 97%, e.g., a diastereomeric and enantiomeric excess equal to or greater than 98%, e.g., a diastereomeric and enantiomeric excess equal to or greater than 99%.
1.7 Any of Method 1 or 1.1-1.6, wherein the compound is in free form.
1.8 Method 1.3, wherein the method comprises administering an effective amount of Compound B:

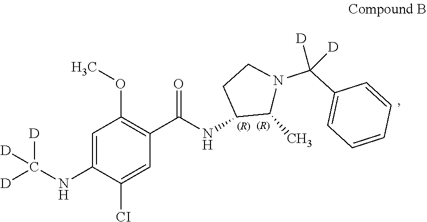

in free or pharmaceutically acceptable salt form, e.g., in free form.
1.9 Method 1.8, wherein the effective amount of Compound B, in free or pharmaceutically acceptable salt form, has a stereoisomeric excess of greater than 90%, e.g., a stereoisomeric excess equal to or greater than 95%, e.g., a stereoisomeric excess equal to or greater than 96%, e.g., a stereoisomeric excess equal to or greater than 97%, e.g., a stereoisomeric excess equal to or greater than 98%, e.g., a stereoisomeric excess equal to or greater than 99%. For instance, wherein the effective amount of Compound B, in free or pharmaceutically acceptable salt form, is substantially diastereomerically and/or enantiomerically pure, e.g., wherein the effective amount of Compound B, in free or pharmaceutically acceptable salt form, is substantially diastereomerically and enantiomerically pure. For instance, wherein the effective amount of compound B, in free or pharmaceutically acceptable salt form, has a diastereomeric and/or enantiomeric excess of greater than 90%, e.g., a diastereomeric and/or enantiomeric excess equal to or greater than 95%, e.g., a diastereomeric and/or enantiomeric excess equal to or greater than 96%, e.g., a diastereomeric and/or enantiomeric excess equal to or greater than 97%, e.g., a diastereomeric and/or enantiomeric excess equal to or greater than 98%, e.g., a diastereomeric and/or enantiomeric excess equal to or greater than 99%. For instance, wherein the effective amount of compound B, in free or pharmaceutically acceptable salt form, has a diastereomeric and enantiomeric excess of greater than 90%, e.g., a diastereomeric and enantiomeric excess equal to or greater than 95%, e.g., a diastereomeric and enantiomeric excess equal to or greater than 96%, e.g., a diastereomeric and enantiomeric excess equal to or greater than 97%, e.g., a diastereomeric and enantiomeric excess equal to or greater than 98%, e.g., a diastereomeric and enantiomeric excess equal to or greater than 99%.
1.10 Any of Method 1 or 1.3-1.9, wherein the designation of deuterium (i.e., D) at a position means that position has a significantly greater than natural abundance of deuterium at that position (e.g., greater than 0.1%, or greater than 0.5%, or greater than 1%, or greater than 5%). Any atom not designated as a particular isotope is present at natural isotopic abundance.
1.11 Any of Method 1 or 1.3-1.10, wherein the compound, in free or pharmaceutically acceptable salt form, has greater than 50% incorporation of deuterium (i.e., D) at one or more positions (e.g., at all positions) designated as deuterium (i.e., D), e.g., greater than 60%, or greater than 70%, or greater than 80%, or greater than 90%, or greater than 95%, or greater than 96%, or greater than 97%, or greater than 98%, or greater than 99%. For instance, any of Method 1 or 1.3-1.10, wherein the compound, in free or pharmaceutically acceptable salt form, has greater than 50% incorporation of deuterium (i.e., D) at each position designated as deuterium (i.e., D), e.g., greater than 60%, or greater than 70%, or greater than 80%, or greater than 90%, or greater than 95%, or greater than 96%, or greater than 97%, or greater than 98%, or greater than 99%.
1.12 Any of Method 1 or 1.1-1.11, wherein the disorder is a brain disorder. For instance, any of Method 1 or 1.1-1.11, wherein the disorder is a neuropsychiatric condition in which anhedonia is prominent.
1.13 Any of Method 1 or 1.1-1.12, wherein the disorder is an affective (mood) disorder or an anxiety disorder.
1.14 Any of Method 1 or 1.1-1.13, wherein the disorder is depression (e.g., depression associated with anhedonia), an anxiety disorder, psychosis (e.g., psychosis in neurodegenerative conditions, such as psychosis in Alzheimer's disease, Parkinson's disease, or dementia (e.g., dementia-related psychosis)), schizophrenia, schizoaffective disorder, post-traumatic stress disorder (PTSD), attention-deficit/hyperactivity disorder (ADHD), Tourette syndrome, anorexia nervosa, bulimia nervosa, binge-eating disorder, body dysmorphic disorder, obsessive compulsive disorder, addiction, bipolar disorder (including bipolar depression, bipolar mania, and bipolar disorder with mixed features), or a migraine. For instance, any of Method 1 or 1.1-1.13, wherein the anxiety disorder is panic disorder, social anxiety disorder, a phobia, or generalized anxiety disorder. Or, any of Method 1 or 1.1-1.13, wherein the method is prophylaxis or treatment of behavioral and psychological symptoms of dementia including agitation, depression, anxiety, apathy, and/or psychosis.
1.15 Any of Method 1 or 1.1-1.14, wherein the disorder is anhedonia or depression associated with anhedonia, suicidal ideation, anxious depression, inflammatory depression, treatment-resistant depression, dysthymia, bipolar depression, psychotic depression, or post-psychotic depression. For instance, any of Method 1 or 1.1-1.14, wherein the disorder is anxious depression. Or, for instance any of Method 1 or 1.1-1.14, wherein the disorder is melancholic depression.
1.16 Any of Method 1 or 1.1-1.15, wherein the disorder is major depressive disorder.
1.17 Any of Method 1 or 1.1-1.14, wherein the disorder is a substance use disorder.
1.18 Any of Method 1 or 1.1-1.14, wherein the method is prophylaxis or treatment of negative symptoms of schizophrenia. Or, any of Method 1 or 1.1-1.14, wherein the method is improving cognition in schizophrenia.
1.19 Any of Method 1 or 1.1-1.11, wherein the compound, in free or pharmaceutically acceptable salt form, is administered as an anti-emetic.
1.20 Any of Method 1 or 1.1-1.19, wherein the method comprises administering 9-60 mg a day of the compound, in free or pharmaceutically acceptable salt form (i.e., 9-60 mg total daily dose of the compound, in free or pharmaceutically acceptable salt form). For instance, any of Method 1 or 1.1-1.19, wherein the method comprises administering 9-36 mg a day of the compound, in free or pharmaceutically acceptable salt form (i.e., 9-36 mg total daily dose of the compound, in free or pharmaceutically acceptable salt form).
1.21 Any of Method 1 or 1.1-1.20, wherein the method comprises administering an amount of the compound, in free or pharmaceutically acceptable salt form, that provides 55%-80% D2/D3 receptor occupancy, e.g., as measured by positron emission tomography. For instance, wherein the method comprises administering an amount of the compound, in free or pharmaceutically acceptable salt form, that provides about 65% D2/D3 receptor occupancy, e.g., as measured by positron emission tomography. Or, for instance, wherein the method comprises administering an amount of the compound, in free or pharmaceutically acceptable salt form, that provides about 60% D2/D3 receptor occupancy, e.g., as measured by positron emission tomography.
1.22 Method 1.20 or 1.21, wherein the disorder is psychosis (e.g., psychosis in neurodegenerative conditions, such as Alzheimer's disease, Parkinson's disease, and dementia (e.g., dementia-related psychosis)), schizophrenia, schizoaffective disorder, or bipolar disorder (e.g., bipolar mania).
1.23 Method 1.20 or 1.21, wherein the method is prophylaxis or treatment of negative symptoms of schizophrenia. Or, Method 1.20 or 1.21, wherein the method is improving cognition in schizophrenia.
1.24 Any of Method 1 or 1.1-1.19, wherein the method comprises administering 1-9 mg (e.g., 1-8 mg, e.g., 1.5-6 mg) a day of the compound, in free or pharmaceutically acceptable salt form (i.e., 1-9 mg total daily dose, e.g., 1-8 mg total daily dose, e.g., 1.5-6 mg total daily dose, of the compound, in free or pharmaceutically acceptable salt form). For instance, any of Method 1 or 1.1-1.19, wherein the method comprises administering 1-8 mg a day of the compound, in free or pharmaceutically acceptable salt form (i.e., 1-8 mg total daily dose of the compound, in free or pharmaceutically acceptable salt form). For instance, any of Method 1 or 1.1-1.19, wherein the method comprises administering 1-3 mg a day of the compound, in free or pharmaceutically acceptable salt form (i.e., 1-3 mg total daily dose of the compound, in free or pharmaceutically acceptable salt form). For instance, any of Method 1 or 1.1-1.19, wherein the method comprises administering 1 mg to less than 3 mg a day (e.g., 2 mg a day) of the compound, in free or pharmaceutically acceptable salt form (i.e., 1 mg to less than 3 mg total daily dose of the compound, in free or pharmaceutically acceptable salt form).
1.25 Any of Method 1, 1.1-1.19, or 1.24, wherein the method comprises administering an amount of the compound, in free or pharmaceutically acceptable salt form, that provides 10%-60% (e.g., 40%-60% or, e.g., 10%-55%, e.g., 10%-50%, e.g., 30%-50% or, e.g., 15%-50%, e.g., 15%-45%, e.g., 20%-40%, e.g., 10%-30%) D2/D3 receptor occupancy, e.g., as measured by positron emission tomography. Or, for instance, any of Method 1, 1.1-1.19, or 1.24, wherein the method comprises administering an amount of the compound, in free or pharmaceutically acceptable salt form, that provides ≤40% (e.g., about 40%), e.g., <40% D2/D3 receptor occupancy, e.g., as measured by positron emission tomography.
1.26 Method 1.24 or 1.25, wherein the disorder is depression (e.g., depression associated with anhedonia), an anxiety disorder, post-traumatic stress disorder (PTSD), attention-deficit/hyperactivity disorder (ADHD), Tourette syndrome, anorexia nervosa, bulimia nervosa, binge-eating disorder, body dysmorphic disorder, obsessive compulsive disorder, addiction, bipolar disorder, bipolar disorder with mixed features, or a migraine. For instance, Method 1.24 or 1.25, wherein the anxiety disorder is panic disorder, social anxiety disorder, a phobia, or generalized anxiety disorder.
1.27 Any of Method 1.24-1.26, wherein the disorder is anhedonia or depression associated with anhedonia, suicidal ideation, anxious depression, inflammatory depression, treatment-resistant depression, dysthymia, bipolar depression, psychotic depression, or post-psychotic depression. For instance, wherein the disorder is anxious depression.
1.28 Any of Method 1.24-1.27, wherein the disorder is major depressive disorder.
1.29 Method 1.24 or 1.25, wherein the disorder is a substance use disorder.
1.30 Any of Method 1 or 1.1-1.29, wherein the method comprises administering a pharmaceutical composition comprising the compound, in free or pharmaceutically acceptable salt form. For instance, any of Method 1 or 1.1-1.29, wherein the method comprises administering Formula 1.13 or any of Composition 1 or 1.1-1.15 vide supra.
1.31 Any of Method 1 or 1.1-1.30, wherein the method comprises administering the compound of Formula Ia, in free or pharmaceutically acceptable salt form, once, twice, or three times a day, e.g., once a day. For instance, any of Method 1 or 1.1-1.30, wherein the method comprises administering a pharmaceutical composition comprising the compound of Formula Ia, in free or pharmaceutically acceptable salt form, once, twice, or three times a day, e.g., once a day.
1.32 Any of Method 1 or 1.1-1.31, wherein the method comprises administering the compound of Formula I, in free or pharmaceutically acceptable salt form, once, twice, or three times a day, e.g., once a day. For instance, any of Method 1 or 1.1-1.31, wherein the method comprises administering a pharmaceutical composition comprising the compound of Formula I, in free or pharmaceutically acceptable salt form, once, twice, or three times a day, e.g., once a day.
1.33 Any of Method 1 or 1.1-1.32, wherein the method comprises administering Compound B, in free or pharmaceutically acceptable salt form, once, twice, or three times a day, e.g., once a day. For instance, any of Method 1 or 1.1-1.32, wherein the method comprises administering a pharmaceutical composition comprising Compound B, in free or pharmaceutically acceptable salt form, once, twice, or three times a day, e.g., once a day.

Further provided is a compound of Formula I (e.g., any of Formula 1.1-1.13) or a pharmaceutical composition disclosed herein (e.g., Formula 1.13 or any of Composition 1 or 1.1-1.15) for use in any of Method 1 or 1.1-1.33 vide supra.

Further provided is use of a compound of Formula I (e.g., any of Formula 1.1-1.13) or a pharmaceutical composition disclosed herein (e.g., Formula 1.13 or any of Composition 1 or 1.1-1.15) in any of Method 1 or 1.1-1.33 vide supra.

Further provided is use of a compound of Formula I (e.g., any of Formula 1.1-1.13) in the manufacture of a medicament (e.g., Formula 1.13 or any of Composition 1 or 1.1-1.15) for use in any of Method 1 or 1.1-1.33 vide supra.

Further provided are intermediate compounds of Formula II and Formula III, each in free or salt (e.g., pharmaceutically acceptable salt) form.

For instance, further provided is a compound of Formula II:

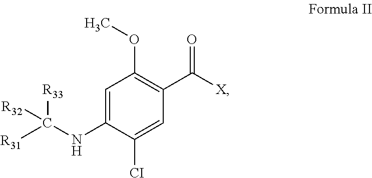

wherein R₃₁, R₃₂, and R₃₃ are independently selected from H and D;
X is OH or a leaving group; and
at least one of R₃₁, R₃₂, and R₃₃ are D;
in free or salt (e.g., pharmaceutically acceptable salt) form.

Further provided are compounds of Formula II as follows:
2.1 Formula II, wherein the compound is in pharmaceutically acceptable salt form.
2.2 Formula II or 2.1, wherein R₃₁, R₃₂, and R₃₃ are D.
2.3 Any of Formula II, 2.1, or 2.2, wherein X is OH.
2.4 Any of Formula II, 2.1, or 2.2, wherein X is a leaving group (e.g., an activated ester, e.g., an O-acylisourea, or a halide). For instance, any of Formula II, 2.1, or 2.2, wherein the compound of Formula II is reacted with 1-ethyl-3-(3-dimethylaminopropyl)carbodiimide.
2.5 Any of Formula II or 2.1-2.3, wherein the compound is:

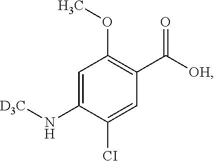

in free or salt (e.g., pharmaceutically acceptable salt) form, e.g., in free form.

Also further provided is a compound of Formula III:

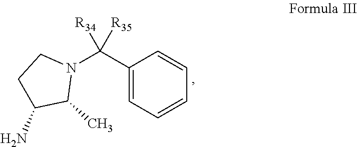

wherein:
R₃₄ and R₃₅ are D;
in free or salt (e.g., pharmaceutically acceptable salt) form.

Further provided are compounds of Formula III as follows:
3.1 Formula III, wherein the compound is in pharmaceutically acceptable salt form.
3.2 Any of Formula III or 3.1, wherein the compound is:

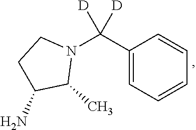 [SMILES: [2H]C([2H])(c1ccccc1)N1CC[C@@H](N)[C@H]1C]

in free or salt (e.g., pharmaceutically acceptable salt) form, e.g., in free form.

Further provided is a process (Process 1) for synthesizing a compound of Formula I (e.g., any of Formula 1.1-1.13), in free or salt (e.g., pharmaceutically acceptable salt) form.

Further provided is Process 1 as follows:
1.1 Process 1, wherein the process comprises reacting a compound of Formula II (e.g., any of Formula 2.1-2.5) with a compound of Formula III (e.g., any of Formula 3.1-3.2).
1.2 Process 1 or 1.1, wherein the process occurs in the presence of an amine (e.g., triethylamine, e.g., triethylamine and dimethylformamide).
1.3 Process 1, 1.1, or 1.2, wherein the process occurs in an organic solvent (e.g., dimethylformamide).
1.4 Any of Process 1 or 1.1-1.3, wherein the process occurs with 1-ethyl-3-(3-dimethylaminopropyl)carbodiimide and hydroxybenzotriazole. For instance, any process wherein the process occurs with 1-ethyl-3-(3-dimethylaminopropyl)carbodiimide, hydroxybenzotriazole, triethylamine, and dimethylformamide.
1.5 Any of Process 1 or 1.1-1.4, wherein the process comprises reacting a compound of Formula IIa:

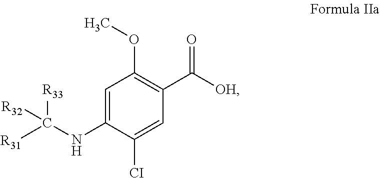

wherein R₃₁, R₃₂, and R₃₃ are independently selected from H and D and at least one of R₃₁, R₃₂, and R₃₃ is D, in free or salt (e.g., pharmaceutically acceptable salt) form, with an activating agent (e.g., 1-ethyl-3-(3-dimethylaminopropyl)carbodiimide).
1.6 Process 1.5, wherein the process forms a compound of Formula IIb:

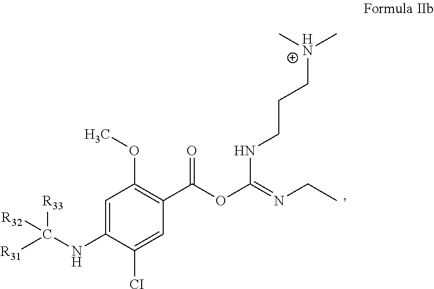

wherein R₃₁, R₃₂, and R₃₃ are independently selected from H and D and at least one of R₃₁, R₃₂, and R₃₃ is D, in free or salt (e.g., pharmaceutically acceptable salt) form.
1.7 Process 1.6, wherein the compound of Formula IIb is formed in situ.

For compounds disclosed herein, a hydrogen atom position of a structure is considered substituted with deuterium when the abundance of deuterium at that position is enriched. The natural abundance of deuterium is about 0.02%, so a compound is “enriched” with deuterium at a specific position when the frequency of incorporation of deuterium at that position exceeds 0.02%. Therefore, for deuterated compounds disclosed herein, any position designated as deuterium (i.e., D) may be enriched with deuterium at a level of greater than 0.1%, or greater than 0.5%, or greater than 1%, or greater than 5%, such as, greater than 50%, or greater than 60%, or greater than 70%, or greater than 80%, or greater than 90%, or greater than 95%, or greater than 96%, or greater than 97%, or greater than 98%, or greater than 99%. For compounds disclosed herein, any atom not designated as a particular isotope is present at natural isotopic abundance.

Compounds disclosed herein, e.g., any of Formula I (e.g., any of Formula 1.1-1.13), Formula Ia, Formula II (e.g., any of Formula 2.1-2.5), Formula III (e.g., any of Formula 3.1-3.2), Formula IIa, Formula IIb, Compound A, and Compound B, may exist in free or salt form, e.g., as acid addition salts. As used herein, unless otherwise indicated, language such as “compound of formula” is to be understood as embracing the compound in any form, for example free or acid addition salt form, or where the compound contains an acidic substituent, in base addition salt form. Compounds of Formula I (e.g., any of Formula 1.1-1.13), Formula Ia, Compound A, and Compound B are intended for use as pharmaceuticals, therefore pharmaceutically acceptable salts are preferred. Salts which are unsuitable for pharmaceutical uses may be useful, for example, for the isolation or purification of free compounds of Formula I or Formula Ia or their pharmaceutically acceptable salts, so therefore are also included.

Isolation or purification of the stereoisomers of compounds disclosed herein, for instance, Formula I (e.g., any of Formula 1.1-1.13), Formula Ia, Formula II (e.g., any of Formula 2.1-2.5), Formula III (e.g., any of 3.1-3.2), Formula IIa, Formula IIb, Compound A, and Compound B, any in free or pharmaceutically acceptable salt form, may be achieved by conventional methods known in the art, e.g., column purification, preparative thin layer chromatography, preparative HPLC, trituration, simulated moving beds, and the like.

Pure stereoisomeric forms of the compounds and intermediates disclosed herein are isomers substantially free of other enantiomeric and diastereomeric forms of the same basic molecular structure of said compounds or intermediates. “Substantially stereoisomerically pure” includes compounds or intermediates having a stereoisomeric excess of greater than 90% (i.e., more than 90% of one isomer and less than 10% of any other possible isomer). The terms “substantially diastereomerically pure” and “substantially enantiomerically pure” should be understood in a similar way, but then having regard to the diastereomeric excess and enantiomeric excess, respectively, of the material in question.

Compounds disclosed herein, e.g., any of Formula I (e.g., any of Formula 1.1-1.13), Formula Ia, Formula II (e.g., any of Formula 2.1-2.5), Formula III (e.g., any of 3.1-3.2), Formula IIa, Formula IIb, Compound A, and Compound B, any in free or pharmaceutically acceptable salt form, may be made by using the methods as described and exemplified herein and by methods similar thereto and by methods known in the chemical art. Such methods include, but are not limited to, those described below. If not commercially available, starting materials for these processes may be made by procedures, which are selected from the chemical art using techniques that are similar to or analogous to the synthesis of known compounds.

Pharmaceutically acceptable salts of any of Formula I (e.g., any of Formula 1.1-1.13), Formula Ia, Formula II (e.g., any of Formula 2.1-2.5), Formula III (e.g., any of 3.1-3.2), Formula IIa, Formula IIb, Compound A, and Compound B, may be synthesized from the parent compound which contains a basic or acidic moiety by conventional chemical methods. Generally, such salts can be prepared by reacting the free base forms of these compounds with a stoichiometric amount of the appropriate acid in an appropriate solvent.

For methods of treatment, the word “effective amount” is intended to encompass a therapeutically effective amount to treat a specific disease or disorder.

Dosages employed in practicing the present invention will of course vary depending, e.g. on the particular disease or condition to be treated, the particular compound used, the mode of administration, and the therapy desired.

Compounds disclosed herein, e.g., any of Formula I (e.g., any of Formula 1.1-1.13), Formula Ia, Compound A, or Compound B, any in free or pharmaceutically acceptable salt form, may be administered by any suitable route, including orally, parenterally, or transdermally, but are preferably administered orally.

Pharmaceutical compositions comprising compounds disclosed herein, e.g., any of Formula I (e.g., any of Formula 1.1-1.13 or any of Composition 1 or 1.1-1.15), Formula Ia, Compound A, or Compound B, any in free or pharmaceutically acceptable salt form, may be prepared using conventional diluents or excipients and techniques known in the galenic art. Thus oral dosage forms may include tablets, capsules, solutions, suspensions, and the like.

### EXAMPLES

### Abbreviations

AcOH=acetic acid
Boc=tert-butyloxycarbonyl
DIAD=diisopropyl azodicarboxylate
DCM=dichloromethane
DMAP=4-dimethylaminopyridine
DMF=dimethylformamide
EDCI=1-ethyl-3-(3-dimethylaminopropyl)carbodiimide
EtOAc or EA=ethyl acetate
h=hour(s)
HOBt=hydroxybenzotriazole
MeOH=methanol
MsCl=methanesulfonyl chloride
rt (or RT)=room temperature
TEA=triethylamine
TFA=trifluoroacetic acid
THF=tetrahydrofuran

### Example 1

### Synthesis of A2: 5-Chloro-N-((2R,3R)-1-(dideutero(phenyl)methyl)-2-methylpyrrolidin-3-yl)-2-methoxy-4-(trideuteromethylamino)benzamide

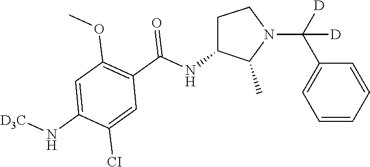

### Compound 15: tert-butyl (R)-2-methyl-3,5-dioxopyrrolidine-1-carboxylate

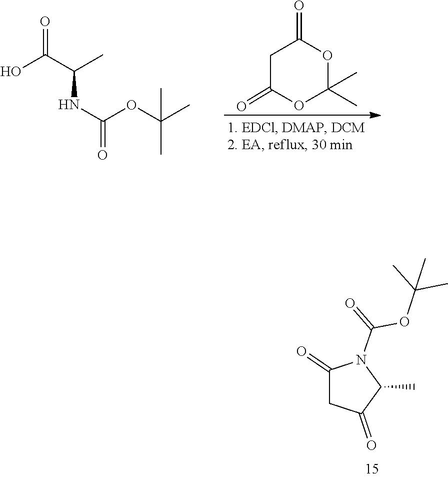

To a stirred solution of Boc-L-alanine (500 g, 2.64 mol), Meldrum's acid (400 g, 2.78 mol) and DMAP (388 g, 3.18 mol) in CH₂Cl₂(5 L) is added EDCI (608 g, 3.18 mol) under nitrogen at 0° C. The resulting solution is then allowed to warm up to room temperature (rt) and stirred over 16 h. It is quenched with water (1.5 L), the organic phase is washed with a cold solution of 5% KHSO₄ (3 L×3), water (3 L×1), and brine, then dried over anhydrous MgSO₄, and concentrated to give the residue. EtOAc (4 L) is added and the reaction mixture is refluxed for 2 hours. The solution is concentrated and the residue is stirred in EtOAc (1 L) at −10° C. for 2 h, then filtered, and the filter cake is collected to give the title compound as a white solid (150 g, 27% yield). The mother liquid is further refluxed for 2 hours then stirred in EA at −10° C. and filtered to give the title compound (40 g) as a white solid. ¹H NMR (400 MHz, CDCl₃) δ 4.45 (q, J=6.8 Hz, 1H), 3.22 (s, 2H), 1.57 (s, 9H), 1.51 (d, J=6.8 Hz, 3H). MS m/z (ESI): 158 [M+H-56]⁺

### Compound 16: tert-butyl (2R,3R)-3-hydroxy-2-methyl-5-oxopyrrolidine-1-carboxylate

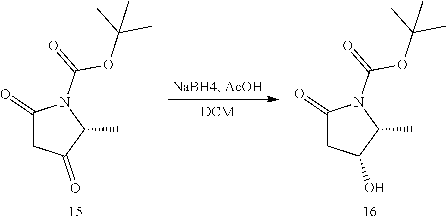

To a stirred solution of compound 15 (40 g, 187.6 mmol) in DCM (400 ml) is added AcOH (200 mL) at 0° C., then NaBH₄ (21.3 g, 562.8 mmol) is added in three portions. The resulting solution is then allowed to warm up to room temperature and stirred over 16 h. The reaction mixture is quenched with 5% NaHCO₃ at 0° C. It is extracted with DCM (200 mL×3). The combined organic layer is washed with 5% NaHCO₃ solution. The organic phase is dried over anhydrous MgSO₄ and concentrated to give the residue that is stirred in isopropyl ether and filtered to give the title compound 16 (24 g, 59.4% yield). ¹H NMR (400 MHz, CDCl₃) δ 4.53-4.47 (m, 1H), 4.29-4.22 (m, 1H), 2.75-2.55 (m, 2H), 1.53 (s, 9H), 1.31 (d, J=6.8 Hz, 3H). MS m/z (ESI): 160 [M+H-56]⁺

### Compound 17: tert-butyl (2R,3R)-3-hydroxy-2-methylpyrrolidine-1-carboxylate

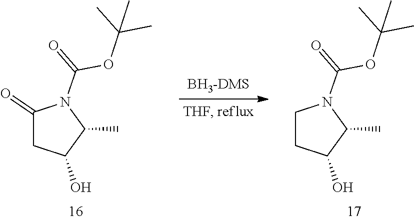

To a solution of compound 16 (87 g, 405 mmol) in dry THE (1 L) is added a solution of BH₃—SMe₂ (600 mL, 1200 mmol) at 0° C. and it is stirred for 30 min at 0° C. Then the mixture is refluxed for 4 h. The resulting mixture is cooled and quenched with saturated NH₄Cl at 0° C. It is then extracted with EtOAc (1 L×3). The organic phases are dried over anhydrous MgSO₄ and concentrated to give compound 17 (70 g, 86% yield). ¹H NMR (400 MHz, DMSO-d6) δ 5.11 (s, 1H), 4.19-4.10 (m, 1H), 3.83-3.63 (m, 1H), 3.22-2.89 (m, 2H), 1.87-1.54 (m, 2H), 1.38 (s, 9H), 0.85 (d, J=6.8 Hz, 3H). MS m/z (ESI): 146 [M+H-56]⁺

### Compound 18

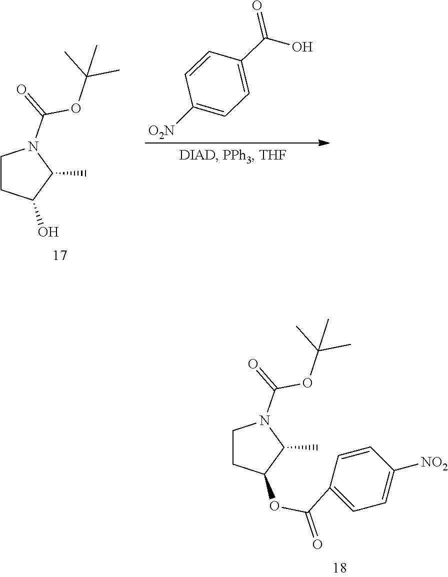 [SMILES: C[C@@H]1[C@H](O)CCN1C(=O)OC(C)(C)C ; O=C(O)c1ccc([N+](=O)[O-])cc1 ; C[C@@H]1[C@@H](OC(=O)c2ccc([N+](=O)[O-])cc2)CCN1C(=O)OC(C)(C)C]

To a cold solution of compound 17 (15.74 g, 78.2 mmol), 4-nitrobenzoic acid (13.72 g, 82.1 mmol), and PPh₃ (16.42 g, 62.6 mmol) in dry THE (250 ml) is added DIAD (16.6 g, 82.1 mmol) for 30 minutes at 0° C. The reaction mixture is allowed to warm room temperature for 16 h. The resulting mixture is cooled and quenched with water. The mixture is extracted with EtOAc (200 ml×3), dried over anhydrous MgSO₄, and then concentrated. The residue is purified by silica gel chromatography to afford the compound 18 (24.7 g, 90.1% yield). ¹H NMR (400 MHz, CDCl₃) δ 8.31-8.17 (m, 4H), 5.20 (d, J=4 Hz, 1H), 4.17-3.86 (m, 1H), 3.59-3.46 (m, 2H), 2.35-2.11 (m, 2H) 1.48 (s, 9H), 1.28 (d, J=6.8 Hz, 3H). MS m/z (ESI): 295 [M+H-56]⁺

### Compound 19: (2R,3S)-2-methylpyrrolidin-3-yl 4-nitrobenzoate

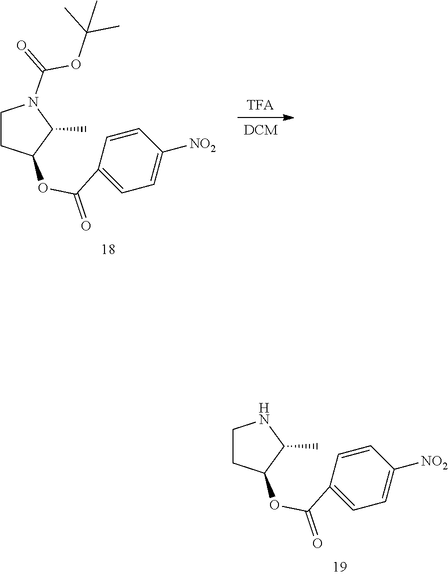 [SMILES: C[C@@H]1[C@@H](OC(=O)c2ccc([N+](=O)[O-])cc2)CCN1C(=O)OC(C)(C)C ; C[C@H]1NCC[C@@H]1OC(=O)c1ccc([N+](=O)[O-])cc1]

A mixture of compound 18 (23.4 g, 66.8 mmol) and TFA (120 mL) in DCM (240 mL) is stirred at room temperature for 1 and then it is concentrated to give compound 19 (16.7 g, 100% yield). LCMS: M+1=251

### Compound 20: (2R,3S)-1-benzoyl-2-methylpyrrolidin-3-yl 4-nitrobenzoate

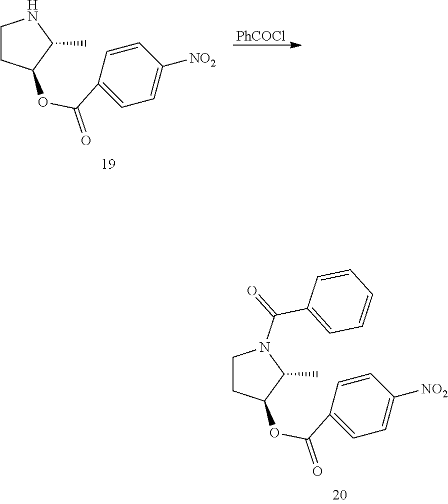 [SMILES: "PhCOCl" ; C[C@H]1NCC[C@@H]1OC(=O)c1ccc([N+](=O)[O-])cc1 ; C[C@@H]1[C@@H](OC(=O)c2ccc([N+](=O)[O-])cc2)CCN1C(=O)c1ccccc1]

To a solution of compound 19 (16.7 g, 66.8 mmol) in toluene (520 ml) is added 2N NaOH (520 ml, 104 mmol), then benzoyl chloride (9.6 g, 66.8 mmol) in toluene (200 ml) is added at 0° C. The mixture is separated and the aqueous phase is extracted with DCM (250 ml×3). The combined organic phase is dried over MgSO₄ and concentrated to give compound 20 (21.3 g, 90% yield). LCMS: M+1=355

### Compound 21: ((2R,3S)-3-hydroxy-2-methylpyrrolidin-1-yl)(phenyl)methanone

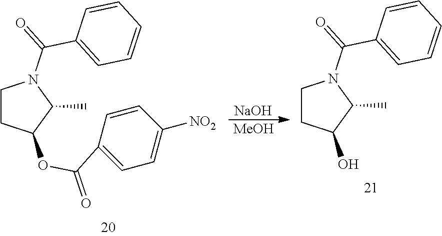

To a stirred solution of compound 20 (21.3 g, 60.1 mmol) in MeOH (400 mL) is added 6N NaOH (11 ml, 66 mmol). The reaction mixture is stirred for 40 minutes and concentrated under reduced pressure. The residue is diluted with DCM (200 ml) and water (100 ml). The aqueous phase is extracted with DCM (250 ml×3). The organic phase is dried over MgSO₄ and concentrated to give the residue that is treated with petroleum ether (10 ml) and EtOAc (2 ml) to afford the title compound 21 (9.85 g, 79.8% yield). LCMS: M+1=206

### Compound 22: (2R,3S)-2-methyl-1-(phenylmethyl-d2)pyrrolidin-3-ol

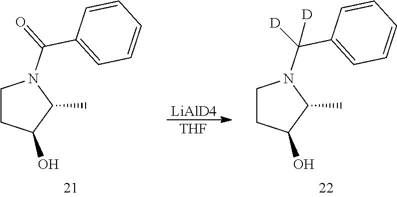

To a stirred solution of compound 21 (3 g, 14.6 mmol) in dry THE (50 mL) is slowly added lithium aluminum deuteride (614 mg, 14.6 mmol) in portions at −15° C. The reaction mixture is then warmed and stirred at room temperature for 18 h. The mixture is cooled to 0° C. and quenched with 20% aqueous KOH (3.5 mL). The mixture is filtered and the precipitate washed with diethyl ether. The combined organic phases are dried over Na₂SO₄ and concentrated to give the residue that is purified by silica gel chromatography to afford the title compound 22 (2.68 g, 95% yield). LCMS: M+1=194

### Compound 23: (2R,3R)-3-azido-2-methyl-1-(phenylmethyl-d2)pyrrolidine

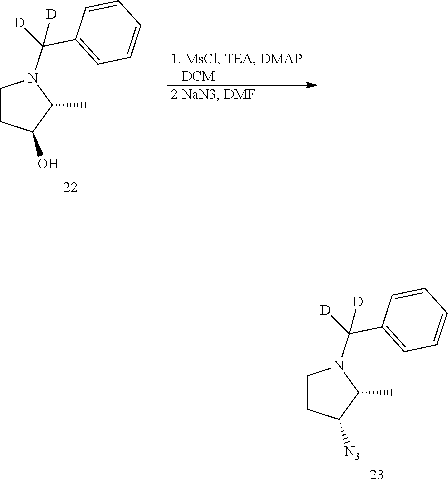

To a stirred solution of compound 22 (1.5 g, 7.8 mmol), DMAP (2.36 g, 0.78 mmol), and Et₃N (95 mg, 23.4 mmol) in dry CH₂Cl₂ at 0° C. is added MsCl (1.8 g, 15.62 mmol). The reaction mixture is stirred for 3 h at room temperature, then quenched with saturated aqueous NaHCO₃, and the aqueous layer extracted with CHCl₃ (30 mL×3). The combined organic phase is washed with brine and dried over anhydrous Na₂SO₄. The organic phase is concentrated under reduced pressure and the residue is diluted with DMF (40 ml) and NaN₃ (1.65 g, 23.4 mmol) is added. The reaction mixture is stirred for 16 h at 80° C. It is quenched with water, extracted with EtOAc (100 ml×3). The organic phase is dried over MgSO₄ and concentrated to give the residue that is purified by silica gel column chromatography to afford the title compound 23 (1.7 g, 99% yield). LCMS: M+1=219

### Compound 24

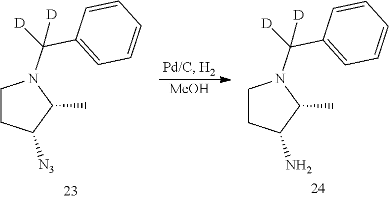 [SMILES: "MeOH" ; [2H]C([2H])(c1ccccc1)N1CC[C@@H](N=[N+]=[N-])[C@H]1C ; "Pd/C, H2" ; [2H]C([2H])(c1ccccc1)N1CC[C@@H](N)[C@H]1C]

A mixture of compound 23 (1.36 g, 6.2 mmol) and 10% of Pd/C (151 mg) in MeOH (50 mL) is stirred under H₂ for 18 hours. The reaction mixture is filtered and the solvent evaporated under reduced pressure to afford the compound 24 (1.12 g, 93% yield). LCMS: M+1=193

### Compound 11: Methyl 4-(tert-butoxycarbonylamino)-5-chloro-2-methoxybenzoate

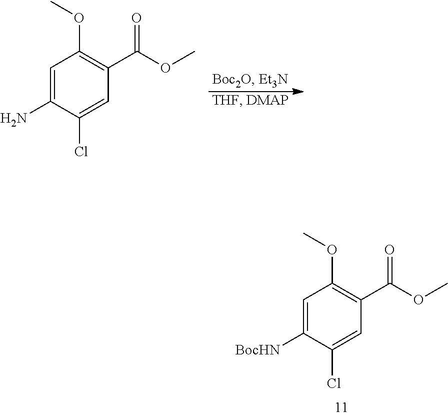

To a mixture of methyl 4-amino-5-chloro-2-methoxybenzoate (5 g, 23.2 mmol), DMAP (1.4 g, 11.7 mmol), and triethylamine (13.2 mL) in THE (175 mL) is added Boc₂O (5.6 g, 25.5 mmol) and the reaction mixture is stirred at 38° C. for 6 h. It is concentrated to give the residue that is purified by silica gel column chromatography to afford the compound 11 (4.4 g, 60% yield). ¹H NMR (400 MHz, CDCl₃) δ 8.71 (s, 1H), 7.72 (s, 1H), 7.62 (s, 1H), 3.81 (s, 3H), 3.77 (s, 3H), 1.49 (s, 9H). MS m/z (ESI): 316 [M+H-56]⁺

### Compound 12: Methyl 4-(tert-butoxycarbonyl(trideuteromethyl)amino)-5-chloro-2-methoxybenzoate

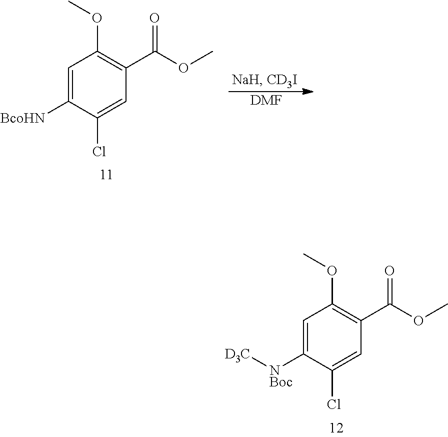

To a solution of compound 11 (1.96 g, 6.2 mmol) in dry DMF (40 mL) is added NaH (370 mg, 9.3 mmol) and the reaction is stirred at room temperature for 30 minutes, then CD₃I (1.8 g, 12.4 mmol) is added and the reaction is stirred at room temperature for 3 h. The reaction is cooled to 0° C. and quenched with saturated aqueous NH₄Cl. It is extracted with EtOAc (100 mL) and the organic phase is washed with water, brine, and dried over anhydrous Na₂SO₄. The organic phase is concentrated under reduced pressure and the residue purified by silica gel column chromatography to afford the title compound 12 (2.02 g, 97.6% yield). NMR (400 MHz, CDCl₃) δ 7.86 (s, 1H), 6.85 (s, 1H), 3.90 (s, 6H), 1.36 (s, 9H). MS m/z (ESI): 277 [M+H-56]⁺

### Compound 13: 4-(tert-Butoxycarbonyl(trideuteromethyl)amino)-5-chloro-2-methoxybenzoic acid

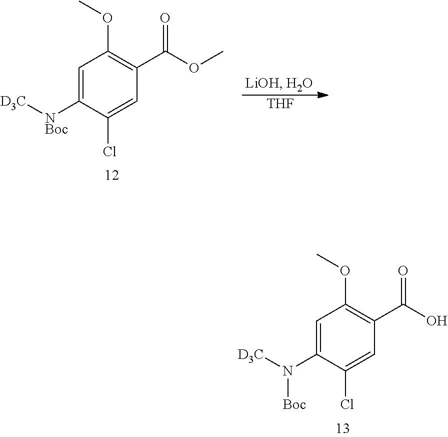

To a solution of compound 12 (1.68 g, 5.1 mmol) in THE (65 mL) and water (20 mL) is added LiOH·H₂O (844 mg, 20 mmol) and the mixture is stirred for 12 h. The reaction is acidified to pH=3 with 1N HCl and then is extracted with EtOAc (50 mL×3) and the combined organic phases are washed with brine and dried over anhydrous Na₂SO₄. The organic phase is concentrated under reduced pressure and the residue is purified by silica gel column chromatography to afford the title compound 13. (1.46 g, 90% yield). ¹H NMR (400 MHz, CDCl₃) δ 8.23 (s, 1H), 6.96 (s, 1H), 4.08 (s, 3H), 1.39 (s, 9H). MS m/z (ESI): 263 [M+H-56]⁺

### Compound 14: 5-Chloro-2-methoxy-4-(trideuteromethylamino)benzoic acid

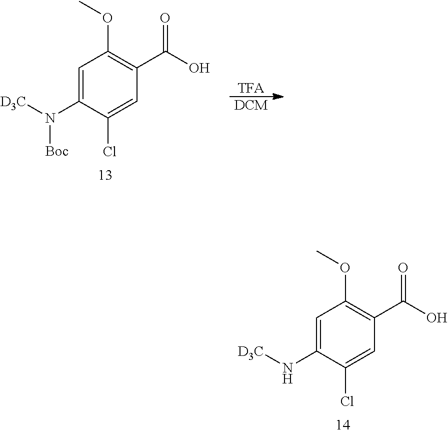

A mixture of compound 13 (1.44 g, 4.5 mmol) and TFA (12 mL) in CH₂Cl₂(25 mL) is stirred at room temperature for 1 h. The reaction mixture is concentrated to give compound 14 (0.73 g, 73.8% yield). ¹H NMR (400 MHz, DMSO-d6) δ 7.61 (s, 1H), 6.20 (s, 1H), 6.18 (s, 1H), 3.83 (s, 3H). MS m/z (ESI): 219 [M+H]⁺

### A2: 5-Chloro-N-((2R,3R)-1-(dideutero(phenyl)methyl)-2-methylpyrrolidin-3-yl)-2-methoxy-4-(trideuteromethylamino)benzamide

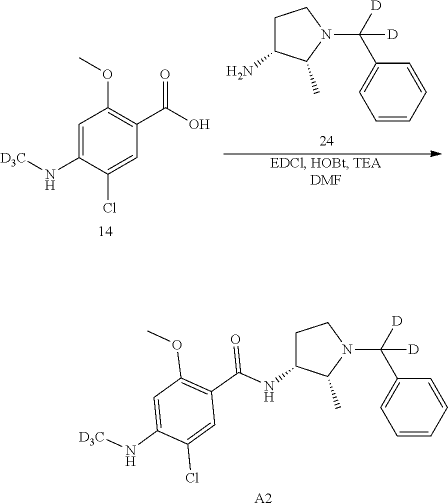 [SMILES: [2H]C([2H])([2H])Nc1cc(OC)c(C(=O)O)cc1Cl ; [2H]C([2H])(c1ccccc1)N1CC[C@@H](N)[C@H]1C ; "EDCl, HOBt, TEADMF" ; [2H]C([2H])([2H])Nc1cc(OC)c(C(=O)N[C@@H]2CCN(C([2H])([2H])c3ccccc3)[C@@H]2C)cc1Cl ; "A2"]

A mixture of compound 24 (100 mg, 0.52 mmol), compound 14 (114 mg, 0.52 mmol), HOBt (105 mg, 0.78 mmol), EDCI (150 mg, 0.78 mmol), and TEA (158 mg, 1.56 mmol) in dry DMF (2 ml) is stirred at room temperature for 16 h. The resulting mixture is quenched with water and extracted with EtOAc (15 mL×3), washed with brine, dried over anhydrous MgSO₄, and concentrated to give the residue which is purified by silica gel chromatography to afford compound A2 (85 mg, 41.5% yield). ¹H NMR (400 MHz, CDCl₃) δ 8.10 (s, 1H), 8.00 (s, 1H), 7.52-7.35 (m, 5H), 6.13 (s, 1H), 4.69 (s, 2H), 4.00 (s, 3H), 2.97 (s, 1H), 2.63 (s, 1H), 2.21-2.05 (m, 2H), 1.59 (s, 1H), 1.13 (s, 3H). MS m/z (ESI): 393 [M+H]⁺

### Example 2—Radioligand Binding Competition Activity on Recombinant Human Dopamine and Serotonin Receptors Using Filtration Binding Assays

Radioligand binding experiments are conducted with membrane preparations. Receptor accession numbers, cellular background, and reference compounds are listed in Table 1.

TABLE 1

| Receptor | Accession Number | Cell Line | Reference Tracer | Reference Competitor |
|---|---|---|---|---|
| D2S | NP_057658.2 | CHO-K1 | [³H]-Spiperone | Risperidone |
| D3 | AAA73929.1 | CHO-K1 | [³H]-R-(+)-7-OH-DPAT | R-(+)-7-OH-DPAT |
| D4.4 | NP_000788.2 | CHO-K1 | [³H]-Spiperone | Haloperidol |
| 5-HT1A | NP_000515.2 | CHO-K1 | [³H]-8-OH-DPAT | 5-HT |
| 5-HT2A | NP_000612.1 | CHO-K1 | [³H]-Ketanserin | Ketanserin |
| 5-HT7A | NP_000863.1 | CHO-K1 | [³H]-LSD | 5-CT |
| human D2L | AAB26819.1 | CHO-K1 | [³H]-Spiperone | Risperidone |

The compound from Example 1 (A2) is tested for radioligand binding competition activity at human Dopamine D2S, D3, and D4.4 and Serotonin 5-HT1A, 5-HT2A, and 5-HT7A receptors and results are provided in Table 2.

TABLE 2
Binding

| Measure | D2S | D3 | D4 | 5-HT1A | 5-HT2A | 5-HT7A | D2L |
|---|---|---|---|---|---|---|---|
| Nemon- | 0.1 | 0.2 | 0.4 | 1.4 | 2.3 | 53.2 | 0.01 |
| apride^{a} (Ki, nM) | | | | | | | |
| Nemon- | 0.5 | 0.3 | 0.8 | 2.7 | 10 | 75.7 | 0.95 |
| apride^{a} (IC₅₀, nM) | | | | | | | |
| cis (R,R) nemon- | 0.2 | 0.5 | 0.8 | 1.4 | 3.3 | 102 | 0.02 |
| apride^{b} (Ki, nM) | | | | | | | |
| cis (R,R) nemon- | 1.00^{c} | 0.8^{c} | 1.6^{c} | 2.8^{c} | 14.5^{c} | 129^{c} | 0.2 |
| apride^{b} (IC₅₀, nM) | (1.26, | (0.7, | (2.25, | (4.7, | (19.4, | (112, | |
| | 0.69) | 0.95) | 0.9) | 0.8) | 9.7) | 145) | |
| Ex. 1 (A2, R,R) | 0.2 | 0.5 | 0.7 | 0.5 | 7.4 | 82.6 | 0.02 |
| Ki (nM) | | | | | | | |
| Ex. 1 (A2, R,R) | 1.0^{c} | 0.8^{c} | 1.3^{c} | 1^{c} | 32.9^{c} | 131^{c} | 0.2 |
| (IC₅₀, nM) | (1.25, | (0.82, | (2.06, | (1.09, | (45.2, | (143, | |
| | 0.69) | 0.85) | 0.59) | 0.91) | 20.5) | 118) | |

^{a}(±)-cis-N-(1-Benzyl-2-methylpyrrolidin-3-yl)-5-chloro-2-methoxy-4-methylaminobenzamide
^{b}N-[(2R,3R)-1-benzyl-2-methylpyrrolidin-3-yl]-5-chloro-2-methoxy-4-(methylamino)benzamide
^{c}Average of numbers in parentheses.

### Example 3—Agonist or Antagonist Activity on Recombinant Human Dopamine and Serotonin Receptors Using IPOne HTRF, cAMP HTRF, and GTPγS Assays

SPA ³⁵S-GTPgS experiments are conducted with membrane preparations. IP-One and cAMP HTRF assays are conducted with recombinant cell lines. Receptor accession numbers, cellular background, and reference compounds are listed in Table 3.

TABLE 3

| Receptor | Accession Number | Assay | Cell Line | Reference Agonist | Reference Antagonist |
|---|---|---|---|---|---|
| D2S | NP_057658.2 | cAMP | CHO-K1 | Quinpirole | Haloperidol |
| D3 | AAA73929.1 | GTP | CHO-K1 | Dopamine | GR103691 |
| D4.4 | NP_000788.2 | cAMP | CHO-K1 | Dopamine | Spiperone |
| 5-HT1A | NP_000515.2 | cAMP | CHO-K1 | 5-CT | Not tested |
| 5-HT2A | NP_000612.1 | IPOne | CHO-K1 | α-Me-5-HT | Ketanserin |
| 5-HT7A | NP_000863.1 | cAMP | CHO-K1 | 5-CT | Risperidone |
| D2L | AAB26819.1 | cAMP | CHO-K1 | Quinpirole | Haloperidol |

The compound from Example 1 (A2) is tested for antagonist activity at human Dopamine D2S, D3, and D4.4 receptors, for agonist activity at human Serotonin 5-HT1A receptor, for agonist and antagonist activity at human Serotonin 5-HT2A receptor, and for antagonist activity at human Serotonin 5-HT7A receptor. Results are in Tables 4 and 5.

Agonist activity of test compounds is expressed as a percentage of the activity of the reference agonist at its EC₁₀₀ concentration. Antagonist activity of the test compound is expressed as a percentage of the inhibition of reference agonist activity at its EC₈₀ concentration.

TABLE 4
Functional Assays

| | | | | 5- | 5- | | |
|---|---|---|---|---|---|---|---|
| | | | | HT1A | HT2A | 5- | 5- |
| Measure | D2 | D3 | D4 | (agonist | (agonist | HT2A | HT7A |
| (IC₅₀, | (antagonist | (antagonist | (antagonist | mode) | mode) | (antagonist | (antagonist |
| nM) | mode) | mode) | mode) | EC₅₀ | EC₅₀ | mode) | mode) |
| Nemon- | cAMP | GTPγS | cAMP | cAMP | IP-one | IP-one | — |
| apride^{a} | 0.3 | 3.0 | 0.9 | 14.3 | 5.5 | 6.5 | |
| | (D2S) | | | | | | |
| | cAMP | | | | | | |
| | 0.08 | | | | | | |
| | (D2L) | | | | | | |
| cis (R,R) | cAMP | GTPγS | cAMP | cAMP | IP-one | IP-one | cAMP |
| nemon- | 0.4^{c} | 2.9^{c} | 1.2^{c} | 33.5^{c} | 3.4^{c} | >3000 | 2474 |
| apride^{b} | (0.61, | (1.3, | (1.41, | (30, | (4.4, | | |
| | 0.27) | 4.5) | 0.9) | 37.1) | 2.9, | | |
| | (D2S) | | | | 4.8, | | |
| | cAMP | | | | 1.6) | | |
| | 0.08 | | | | | | |
| | (D2L) | | | | | | |
| Ex. 1 | cAMP | GTPγS | cAMP | cAMP | IP-one | IP-one | cAMP |
| (A2) | 0.8^{c} | 3.8^{c} | 2.5^{c} | 16.1^{c} | 4.9^{c} | >3000 | >3000 |
| | (1.38, | (3.03, | (4.67, | (19.8, | (4.3, | | |
| | 0.3) | 4.6) | 0.37) | 12.3) | 1.5, | | |
| | (D2S) | | | | 9.1, | | |
| | cAMP | | | | 4.9) | | |
| | 0.08 | | | | | | |
| | (D2L) | | | | | | |

^{a}(±)-cis-N-(1-Benzyl-2-methylpyrrolidin-3-yl)-5-chloro-2-methoxy-4-methylaminobenzamide
^{b}N-[(2R,3R)-1-benzyl-2-methylpyrrolidin-3-yl]-5-chloro-2-methoxy-4-(methylamino)benzamide
^{c}Average of numbers in parentheses.

TABLE 5

| | | | | 5- | 5- | 5- | 5- |
|---|---|---|---|---|---|---|---|
| | D2 | D3 | D4 | HT1A | HT2A | HT2A | HT7A |
| | (antagonist | (antagonist | (antagonist | (agonist | (agonist | (antagonist | (antagonist |
| Measure^{a} | mode) | mode) | mode) | mode) | mode) | mode) | mode) |
| Nemon- | cAMP | GTPγS | cAMP | cAMP | IP-one | IP-one | — |
| apride^{b} | 97 | 126 | 75 | 52 | 48 | 33 | |
| | (D2S) | | | | | | |
| | cAMP | | | | | | |
| | 86 | | | | | | |
| | (D2L) | | | | | | |
| cis (R,R) | cAMP | GTPγS | cAMP | cAMP | IP-one | — | — |
| nemon- | 99^{d} | 99^{d} | 111^{d} | 78^{d} | 44^{d} | | |
| apride^{c} | (105, | (80, | (117, | (93, | (25, 57, | | |
| | 92) | 118) | 105) | 62) | 31, 63) | | |
| | (D2S) | | | | | | |
| | cAMP | | | | | | |
| | 94 | | | | | | |
| | (D2L) | | | | | | |
| Ex. 1 | cAMP | GTPγS | cAMP | cAMP | IP-one | — | — |
| (A2) | 99^{d} | 122^{d} | 100^{d} | 64^{d} | 69^{d} | | |
| | (104, | (130, | (111, | (65, | (53, 82, | | |
| | 93) | 115) | 89) | 62) | 68, 72) | | |
| | (D2S) | | | | | | |
| | 81 | | | | | | |
| | (D2L) | | | | | | |

^{a}Top % Inhibition or Activation at maximal concentration
^{b}(±)-cis-N-(1-Benzyl-2-methylpyrrolidin-3-yl)-5-chloro-2-methoxy-4-methylaminobenzamide
^{c}N-[(2R,3R)-1-benzyl-2-methylpyrrolidin-3-yl]-5-chloro-2-methoxy-4-(methylamino)benzamide
^{d}Average of numbers in parentheses.

As shown above, the deuterated compound of Example 1 is a D2/D3/D4 antagonist, 5-HT1A agonist, and 5-HT2A partial agonist.

### Example 4—In Vitro Metabolism

Study compounds are investigated in pooled cryopreserved human (mixed gender) hepatocytes. The incubations are performed using 5 μM initial concentration and sampling at 0, 60, and 120 minute time points. The samples are analyzed using UPLC-QE-orbitrap-MS. Incubation volume: 300 μl in 48-well plate. Number of cells: 1 million viable cells/ml. Test compound: 5 μM (stock solution in DMSO). Incubation medium: pH 7.4, Bioreclamation IVT in vitro KHB medium. Shaking: 600 rpm. Time points: 0, 60, and 120 minutes with and without cells. Temperature: 37° C. Sampling volume: 60 μl. DMSO content in incubation: 0.5%. Termination of incubations: 2-fold volume of 75% acetonitrile. Control: verapamil disappearance rate.

Sample preparation for hepatocyte samples: Samples are centrifuged for 20 min at 2272×g at room temperature and pipetted to a UPLC-plate for analysis.

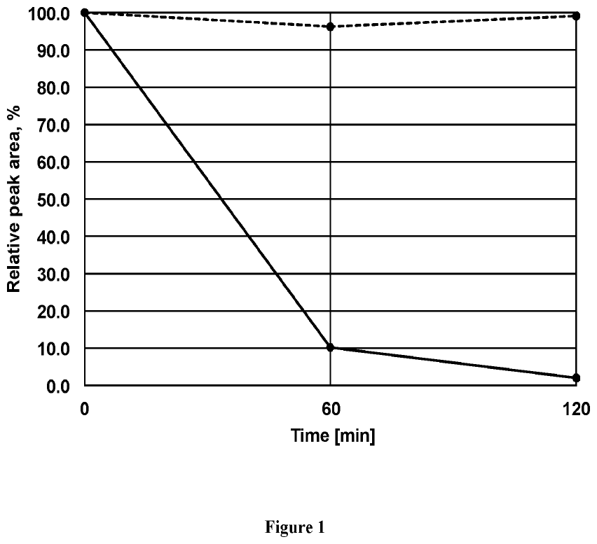

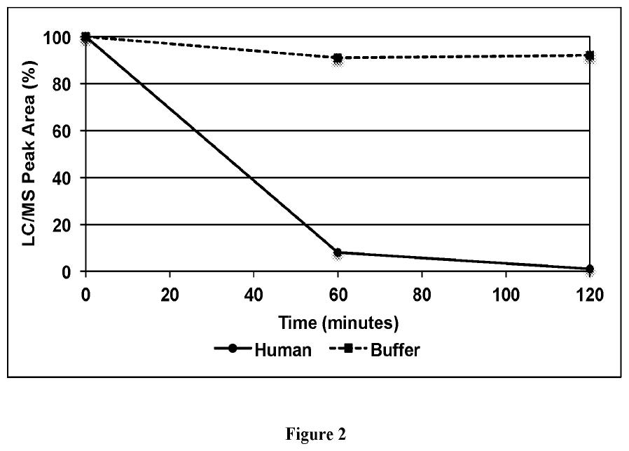

Data are shown in FIGS. 1 and 2 and in the tables below. In both FIGS. 1 and 2, the dashed line is without cells and the solid line is with cells.

TABLE 6

| Compound | 0 min, % | 60 min, % | 120 min, % |
|---|---|---|---|
| 5 μM N-[(2R,3R)-1-benzyl-2-methylpyrrolidin- | 100 | 10 | 2 |
| 3-yl]-5-chloro-2-methoxy-4- | | | |
| (methylamino)benzamide (cis (R,R) | | | |
| nemonapride) | | | |
| 5 μM N-[(2R,3R)-1-benzyl-2-methylpyrrolidin- | 100 | 96 | 99 |
| 3-y1]-5-chloro-2-methoxy-4- | | | |
| (methylamino)benzamide (cis (R,R) | | | |
| nemonapride), no cell control | | | |

TABLE 7

| Compound | Incubation | 0 min, % | 60 min, % | 120 min, % |
|---|---|---|---|---|
| Compound from | Human | 100 | 8 | 1 |
| Example 1 (A2) | Buffer | 100 | 91 | 92 |

### Example 5—In Vivo Pharmacokinetics

Group A male Sprague-Dawley (SD) rats are dosed (by PO) with test compounds at 0.5 mg/kg and 5 mg/kg (N=3 animals/dose level). Blood samples are obtained at 5, 10, and 30 minutes and 1, 2, 4, 8, and 24 hours after dosing. Following blood collection at 24 hours, brain perfusion is performed on the animals before harvesting brain tissues.

Group B male Sprague-Dawley (SD) rats are dosed (by PO) with test compounds at 0.5 mg/kg and 5 mg/kg (N=9 animals/dose level). At designated timepoints (1, 4, and 8 hours), three animals from each dose group undergo blood draw followed by brain perfusion before samples are collected.

Test compounds are the deuterated compound of Example 1 (A2) and N-[(2R,3R)-1-benzyl-2-methylpyrrolidin-3-yl]-5-chloro-2-methoxy-4-(methylamino)benzamide (cis (R,R) nemonapride).

Rats are surgically cannulated with femoral artery catheter for blood collection. Approximate weight of rats is 250-350 g. Water is provided ad libitum. Fasting overnight prior to oral dose. Food available 4 h post dose.

Dose formulations are 0.5% aqueous methylcellulose (4000 cps) with 0.1% Tween™80 for PO administration. Once prepared, the suspension is vortexed/homogenized and continuously stirred until administration. Dose concentration: 0.1 mg/mL for 0.5 mg/kg dose and 1 mg/mL for 5 mg/kg dose. Route of administration: oral gavage. Dose volume: 5 mL/kg. Serial bleed: 200 μL per time point. Terminal bleed: 500 μL.

Blood samples are obtained via an automated sampling system in tubes containing potassium EDTA anticoagulant up to 24 h post dose. Plasma is obtained by centrifugation and snap frozen on dry ice within 30 minutes after collection. Aliquots of each dose formulation are taken, diluted appropriately, and analyzed at the same time with plasma samples by LC-MS/MS.

Plasma (harvested from blood samples) and brain tissues (homogenized and processed) are analyzed by LC/MS/MS. Plasma is harvested from blood via centrifugation within 30 minutes of sample collection. Brain tissue is collected after animals undergo perfusion to remove residual cardiovascular blood.

Dose solutions, plasma (harvested from blood), and brain tissues (homogenized and processed) are stored at −20° C. until analysis.

Plasma samples are thawed at room temperature before adding an organic solvent containing an internal standard to precipitate proteins.

Brain samples are thawed and homogenized in water (3-4 volumes) and aliquots of homogenates analyzed by LC/MS/MS.

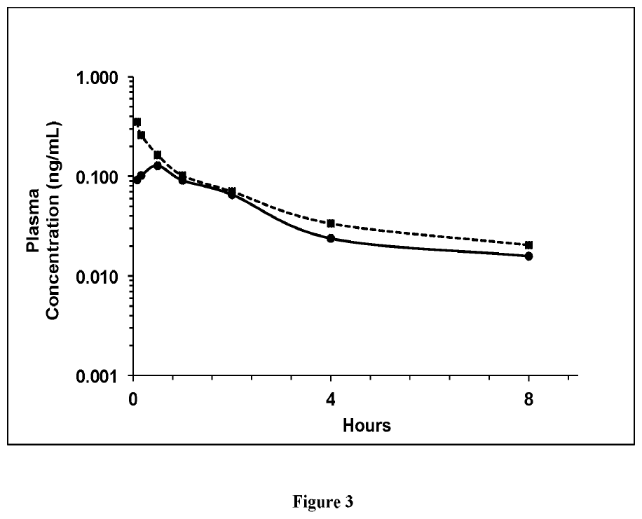

Results are shown in FIGS. 3-6.

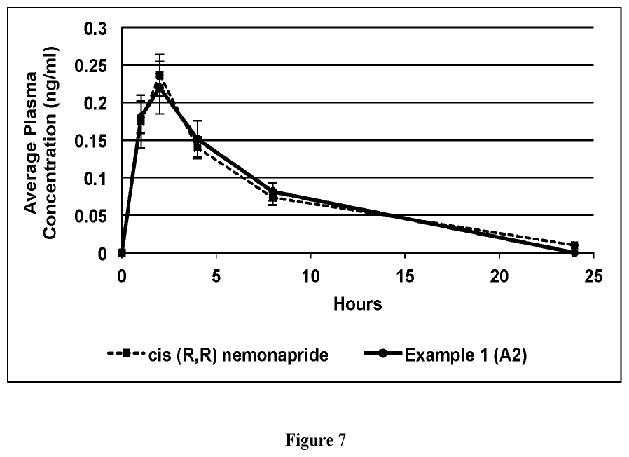

Plasma pharmacokinetics between N-[(2R,3R)-1-benzyl-2-methylpyrrolidin-3-yl]-5-chloro-2-methoxy-4-(methylamino)benzamide (cis (R,R) nemonapride) and the deuterated compound of Example 1 (A2) are similar (see FIG. 3, see also FIG. 7). In FIG. 3, cis (R,R) nemonapride data is shown as the dashed line and average (N=3) data for the deuterated compound of Example 1 (A2) is shown as the solid line.

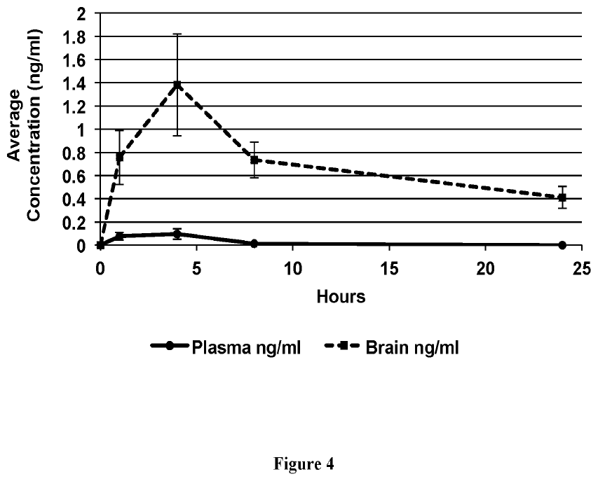

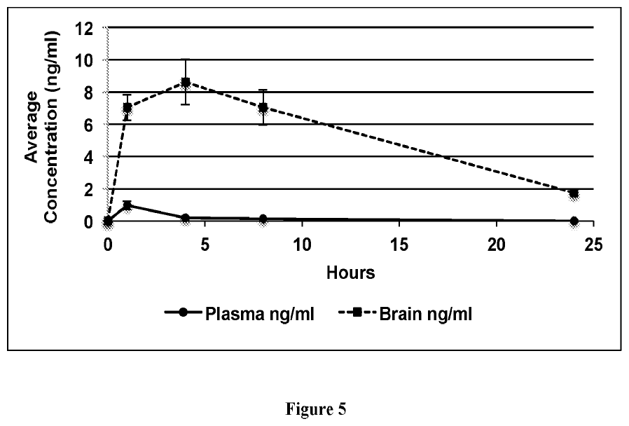

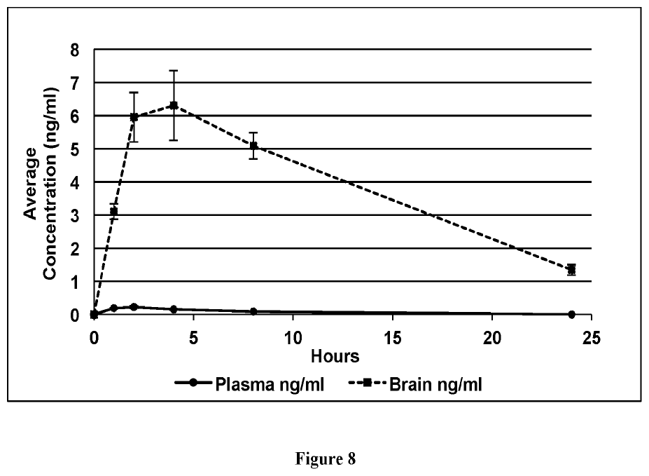

The extended brain enrichment of the compound of Example 1 (A2) in rats following single PO doses of 0.5 mg/kg and 5 mg/kg compared to plasma levels is shown in FIG. 4 and FIG. 5, respectively. In each figure, average brain concentration (ng/ml) is shown as the dashed line and average plasma concentration (ng/ml) is shown as the solid line. While plasma concentrations decrease, brain concentrations increase (see FIG. 5, see also FIG. 8).

The deuterated compound of Example 1 (A2) has enriched brain levels compared to N-[(2R,3R)-1-benzyl-2-methylpyrrolidin-3-yl]-5-chloro-2-methoxy-4-(methylamino)benzamide (cis (R,R) nemonapride) (see FIG. 6, both administered at a single PO dose of 0.5 mg/kg). A comparison of brain to plasma ratios for N-[(2R,3R)-1-benzyl-2-methylpyrrolidin-3-yl]-5-chloro-2-methoxy-4-(methylamino)benzamide (cis (R,R) nemonapride) and the deuterated compound of Example 1 (A2) is in Table 8 (both administered at a single PO dose of 0.5 mg/kg).

TABLE 8

| | cis (R,R) nemonapride | Compound from Example 1 |
|---|---|---|
| Time | Average Brain to | (A2) Average Brain to |
| (hours) | Plasma Partitioning | Plasma Partitioning |
| 1 | 6 | 12 |
| 4 | 17 | 34 |
| 8 | 33 | 56 |
| 24 | ND (plasma values below | ND (plasma values below |
| | quantitation limit) | quantitation limit) |

### Example 6—Ex Vivo Radioligand Binding in Membrane Preparations to Determine Time-Course of Receptor Occupancy at Central D₂ Receptors

This study is to determine receptor occupancy at central D2 receptors following oral administration of the deuterated compound of Example 1 (A2) at various time points (1, 2, 4, 8, and 24 hours) and the positive comparator, olanzapine (10 mg/kg, po) using [³H]raclopride and rat striatal membranes. Liquid scintillation counting is used to quantify radioactivity.

### Animals

Thirty-five male Sprague-Dawley rats. Standard pelleted diet and filtered water is available ad libitum.

### Drug Treatment

On day of test, animals are dosed orally with either vehicle, a single dose (2.5 mg/kg) of the deuterated compound of Example 1 (A2), N-[(2R,3R)-1-benzyl-2-methylpyrrolidin-3-yl]-5-chloro-2-methoxy-4-(methylamino)benzamide (cis (R,R) nemonapride) (2.5 mg/kg), or olanzapine (10 mg/kg, po). Rats are sacrificed at 1 (N=5 rats), 2 (N=5 rats), 4 (N=5 rats), 8 (N=5 rats), and 24 (N=5 rats) hours after drug administration or 1 hour after vehicle and olanzapine administration (N=5 rats for vehicle and N=5 rats for olanzapine). Vehicle is 0.5% methylcellulose.

### Pharmacokinetics

A post-mortem blood sample (approx. 5 ml) is taken by cardiac puncture and placed into K/EDTA tubes. The post-mortem blood samples are gently inverted, centrifuged (1900 g for 5 minutes at 4° C.), and 1 ml of plasma from taken for PK determination. All plasma samples are frozen and stored at −80° C.

Whole brains are removed, rinsed with saline, and blot dried. The left striatum and right striatum is dissected out and weighed before being frozen on dry ice. The striata from each hemisphere are frozen separately. The tissue is wrapped in aluminum foil, placed in bags, and stored at −20° C. until the day of the assay.

### [³H]Raclopride Binding

### Homogenate Preparation

The striata is homogenised individually in ice-cold 50 mM Tris, pH 7.4, 120 mM NaCl, 5 mM KCl, 2 mM CaCl₂, 1 mM MgCl₂, and 10 M pargyline using a tight-fitting homogeniser equivalent to 6.25 mg wet weight of tissue/ml and used immediately in the binding assay.

### Assay

Striatal homogenates (400 μl, equivalent to 2.5 mg wet weight tissue/tube) are incubated with 50 μl of 1.6 nM [³H]raclopride and either 50 μl assay buffer (total binding) or 50 μl of 1 μM (−)sulpiride (to define non-specific binding) for 30 minutes at 23° C. The assay buffer consists of 50 mM Tris, pH 7.4, 120 mM NaCl, 5 mM KCl, 2 mM CaCl₂, 1 mM MgCl₂, and 10 μM pargyline. The wash buffer consists of 50 mM Tris, pH 7.4. There are two tubes for the determination of total binding and two tubes for the determination of non-specific binding.

Membrane bound radioactivity is recovered by filtration under vacuum through filters, presoaked in 0.5% polyethylenimine (PEI) using a cell harvester. Filters are rapidly washed with ice-cold buffer and radioactivity determined by liquid scintillation counting. Data Analysis

A value for specific binding (dpm) is generated by the subtraction of mean non-specific binding (dpm) from mean total binding (dpm) for each animal.

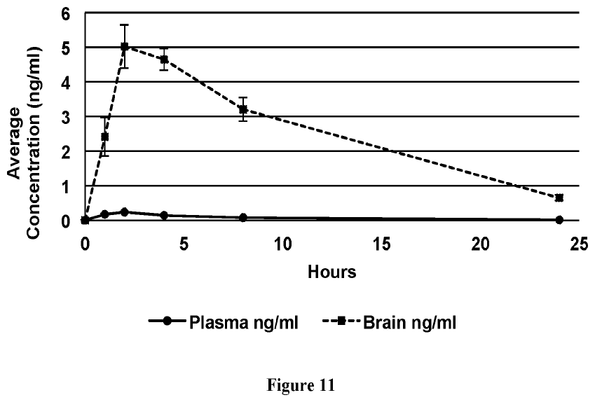

Results are shown in FIGS. 7-11.

Plasma pharmacokinetics between N-[(2R,3R)-1-benzyl-2-methylpyrrolidin-3-yl]-5-chloro-2-methoxy-4-(methylamino)benzamide (cis (R,R) nemonapride) and the deuterated compound of Example 1 (A2) are similar (see FIG. 7, see also FIG. 3). In FIG. 7, average data for cis (R,R) nemonapride data is shown as the dashed line and average data for the deuterated compound of Example 1 (A2) is shown as the solid line (single oral administration of 2.5 mg/kg of each compound).

The extended brain enrichment of the compound of Example 1 (A2) in rats following single oral administration of 2.5 mg/kg compared to plasma levels is shown in FIG. 8. In FIG. 8, average brain concentration (ng/ml) is shown as the dashed line and average plasma concentration (ng/ml) is shown as the solid line.

The deuterated compound of Example 1 (A2) has enriched and retained brain levels compared to N-[(2R,3R)-1-benzyl-2-methylpyrrolidin-3-yl]-5-chloro-2-methoxy-4-(methylamino)benzamide (cis (R,R) nemonapride) (see FIG. 9) (single oral administration of 2.5 mg/kg of each compound). A comparison of brain to plasma ratios for N-[(2R,3R)-1-benzyl-2-methylpyrrolidin-3-yl]-5-chloro-2-methoxy-4-(methylamino)benzamide (cis (R,R) nemonapride) and the deuterated compound of Example 1 (A2) is in Table 9 (single oral administration of 2.5 mg/kg of each compound).

TABLE 9

| | cis (R,R) nemonapride | Compound from Example 1 |
|---|---|---|
| Time | Average Brain to | (A2) Average Brain to |
| (hours) | Plasma Partitioning | Plasma Partitioning |
| 1 | 14 | 18 |
| 2 | 22 | 28 |
| 4 | 35 | 44 |
| 8 | 46 | 66 |
| 24 | ND (most plasma values | ND (plasma values below |
| | below quantitation limit) | quantitation limit) |

In addition, the deuterated compound of Example 1 (A2) has higher receptor occupancy levels at 1 h, 2 h, 8h, and 24h compared to N-[(2R,3R)-1-benzyl-2-methylpyrrolidin-3-yl]-5-chloro-2-methoxy-4-(methylamino)benzamide (cis (R,R) nemonapride) (see FIG. 10).

### Example 7—Touchscreen-Based Rat Probabilistic Reward Task

The Probabilistic Reward Task (PRT) uses visual discrimination methodology to quantify reward responsiveness to both identify deficits and characterize drug-induced improvements. Groups of rats are trained on the touchscreen-based PRT and exposed to asymmetrical probabilistic contingencies to generate response biases to the richly rewarded stimulus (Pizzagalli, D. et al., Biological Psychiatry, 2005, 57, 319-327; Kangas, B. et al., Translational Psychiatry, 2020, 10(1):285; Wooldridge, L. et al., International Journal of Neuropsychopharmacology, 2021, 24, 409-418). Next, subjects are tested with vehicle and three doses of the deuterated compound of Example 1 (A2).

### Methods

### Subjects

Male Sprague Dawley rats are used in the present study.

### Apparatus

Details and schematics of the rodent touch-sensitive experimental chamber can be found in Kangas, B. et al., Behavioural Pharmacology, 2017, 28, 623-629. Briefly, a custom-built Plexiglas chamber (25×30×35 cm) is situated in a sound- and light-attenuating enclosure (40×60×45 cm). A 17″ touch-sensitive screen (1739L, ELO TouchSystems, Menlo Park, CA) comprises the inside right-hand wall of the enclosure. An infusion pump (PHM-100-5, Med Associates, St. Albans, VT) outside the enclosure is used to deliver sweetened condensed milk solution into the shallow reservoir of a custom-designed aluminum receptacle. The receptacle is mounted 3 cm above the floor bars and centered on the left-hand inside wall. Both touchscreen and fluid reservoir are easily accessible to the subject. A speaker bar (NQ576AT, Hewlett-Packard, Palo Alto, CA) mounted above the touchscreen is used to emit audible feedback. All experimental events and data collection are programmed in E-Prime Professional 2.0 (Psychology Software Tools, Inc., Sharpsburg, PA).

### Procedure

### Initial Training

Modified response-shaping techniques are used to train rats to engage with the touchscreen (Kangas, B. et al., Journal of Neuroscience Methods, 2012, 209, 331-336). A 5×5 cm blue square on a black background is presented in different sections of the touchscreen (left, right or center), with the proviso that its lower edge always is 10 cm above the floor bars. This requires the rat to rear on its hind legs to reach the screen and make a touchscreen response with its paw. Each response is reinforced with 0.1 mL of 30% sweetened condensed milk and the delivery is paired with an 880 ms yellow screen flash and 440 Hz tone and followed by a 5-see intertrial interval (ITI) blackout period. After responses are reliably observed with latencies <5 sec following stimulus presentation, line-length discrimination training commenced.

### Line-Length Discrimination Training

Discrete trials begin with concurrent presentation of a white line presented 5 cm above left and right response boxes. The width of the line is always 7 cm, but the length of the line is either 30 cm or 15 cm and varies in a quasi-random fashion across 100-trial sessions (50 trials of each length). Subjects learn to respond to the left or right response box depending on the length of the white line (i.e., long line=respond left, short line=respond right, or vice versa). Response box designation is counter-balanced across subjects. A correct response is reinforced as described above and is followed by a 5 sec ITI, whereas an incorrect response immediately results in a 5 sec ITI. A correction procedure (Kangas, B. et al., Journal of the Experimental Analysis of Behavior, 2008, 90, 103-112) is implemented during initial discrimination training—each incorrect trial is repeated until a correct response is made—and is discontinued after session-wide trial repeats are <5 in each trial type. Discrimination sessions continue without correction until accuracies for both line lengths are >75% correct for 3 consecutive sessions. Probabilistic Reward Task

Following line-length discrimination training, probabilistic reinforcement schedules are introduced. Based on the human task protocol, a 3:1 rich/lean probabilistic schedule is arranged such that 60% of correct responses to one of the line lengths (e.g., long line=rich alternative) and 20% of correct responses to the other line length (e.g., short line=lean alternative) are rewarded. Rich/lean line assignment is counterbalanced across subjects and 50 trials of each trial type are presented in a quasi-random sequence. These probabilistic contingencies are assessed across 5 consecutive sessions prior to initiation of drug testing.

### PRT Drug Tests

Following the establishment of probabilistic contingencies, an acute drug testing protocol is arranged that includes intermittent maintenance sessions in which correct responses on all trials are reinforced, control sessions in which 3:1 (60%:20%) rich/lean probabilistic contingencies are arranged and, no more than once per week, a drug testing session in which vehicle or a dose of the deuterated compound of Example 1 (A2) (0.5, 1, or 2.5 mg/kg) is tested by administering it orally, 4-5 hr prior to a 3:1 (60%:20%) probabilistic session. Doses of the deuterated compound of Example 1 (A2) are tested in a mixed order across subjects using a Latin Square design. Vehicle and all doses of the deuterated compound of Example 1 (A2) are tested in all subjects.

### Data Analysis

The implementation of probabilistic contingencies yields two primary dependent measures: response bias and task discriminability. These can be quantified by examining the number of Correct and Incorrect responses in rich and lean trial types using, respectively, log b and log d equations derived from signal detection theory (Kangas, B. et al., Journal of the Experimental Analysis of Behavior, 2008, 90, 103-112; Luc O. et al., Perspectives on Behavior Science, 2021, 44 (4), 517-540; McCarthy, D., Signal Detection: Mechanisms, Models, and Applications (eds Nevin, J. et al.), Behavioral Detection Theory: Some Implications for Applied Human Research, 1991 (Erlbaum, New Jersey)).

$$log b = 0.5 * log ( \frac{( {Rich}_{Correct} + 0.5 ) * ( {Lean}_{Incorrect} + 0.5 )}{( {Rich}_{Incorrect} + 0.5 ) * ( {Lean}_{Correct} + 0.5 )} ) \\ log d = 0.5 * log ( \frac{( {Rich}_{Correct} + 0.5 ) * ( {Lean}_{Correct} + 0.5 )}{( {Rich}_{Incorrect} + 0.5 ) * ( {Lean}_{Incorrect} + 0.5 )} )$$

High bias values are produced by high numbers of correct responses during rich trials and incorrect responses during lean trials, which increase the log b numerator. High discriminability values are produced by high numbers of correct responses during both rich and lean trials, which increase the log d numerator. (0.5 is added to all parameters to avoid instances where no errors are made on a given trial type, which would make log transformation impossible.) All data (log b, log d, accuracy, reaction time) are subject to repeated measures analysis of variance (ANOVA).

### Drugs

The deuterated compound of Example 1 (A2) is dissolved in a 0.5% methylcellulose solution. Drug doses are administered orally 4-5 hr prior to the experimental session.

### Results and Discussion

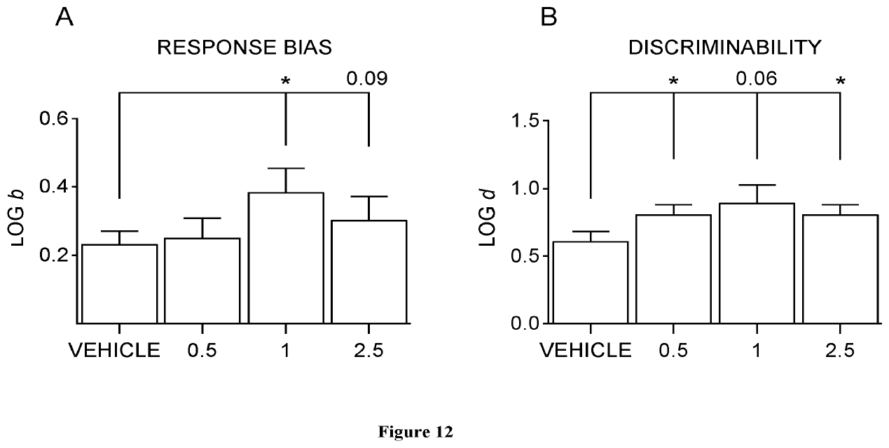

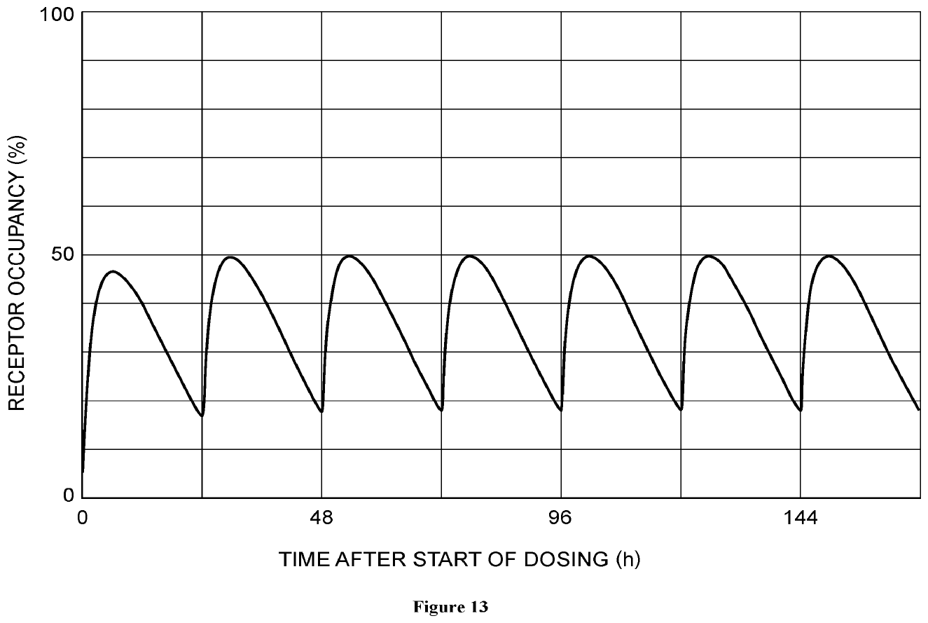

As shown in FIG. 12A, the deuterated compound of Example 1 (A2) enhances reward response bias (log b) significantly at 1 mg/kg.

As shown in FIG. 12B, the deuterated compound of Example 1 (A2) enhances discriminability significantly at 0.5 mg/kg and 2.5 mg/kg, with a trend at 1 mg/kg.

The data shows that a dose of the deuterated compound of Example 1 (A2) targeting low but not high D₂ RO significantly increases reward responsiveness.

No catalepsy is observed at tested doses.

Data shows that the deuterated compound of Example 1 (A2) may reduce anhedonia at low doses without inducing extrapyramidal side effects.

Data suggests that D_{2/3} receptor occupancy of about 40-60% provides anti-anhedonic effects, D_{2/3} receptor occupancy of about 65-80% provides antipsychotic effects, and catalepsy emerges above 80% receptor occupancy.

### Example 8—Conditioned Avoidance Response

Adult male Wistar rats are used. Risperidone (0.5 mg/kg; Sigma Aldrich) is dissolved in 10% DMSO in water and injected i.p. at a dose volume of 1 mg/kg 30 minutes prior to test. The deuterated compound of Example 1 (A2) (0.5, 2.5, and 5 mg/kg) is formulated in 0.5% methyl cellulose in water and administered orally at a dose volume of 1 mg/kg 4 hours prior to test.

The Conditioned Avoidance Response (CAR) Test is an animal model screening for antipsychotic drugs.

Dunnett's post hoc analysis reveals that risperidone (0.5 mg/kg) and the deuterated compound of Example 1 (A2) (2.5 and 5 mg/kg) significantly decrease percent avoidance as well as the number of avoidance responses compared to vehicle.

Dunnett's post hoc analysis reveals that risperidone (0.5 mg/kg) increases escape failures compared to vehicle. None of the doses of the deuterated compound of Example 1 (A2) have a significant treatment effect on this measure.

Rats treated acutely with the deuterated compound of Example 1 (A2) (2.5 and 5 mg/kg) show decreased avoidance responses and percent avoidance indicating potential antipsychotic activity. None of the doses of the deuterated compound of Example 1 (A2) shows any effects on escape failures.

### Example 9—Headshake Response

Adult male Sprague Dawley rats are used. The deuterated compound of Example 1 (A2) (1, 5, and 10 mg/kg) is formulated in 0.5% methylcellulose solution and administered orally (PO) at a dose volume of 1 ml/kg 4 hours prior to test. DOI (3 mg/kg) is dissolved in saline and administered IP at a dose volume of 1 ml/kg (10 minutes prior to test).

Animals are administered vehicle, DOI, or test compound and returned to their holding cage for the appropriate pretreatment time (10 minutes for DOI and 4 hours for the deuterated compound of Example 1 (A2)), following which headshakes are recorded for 10 minutes using video cameras. The headshake response is a rapid, rhythmic shaking of the head in a radial motion. Data are analyzed by ANOVA followed by post hoc analysis where appropriate.

Dunnett's post analysis finds that, compared to vehicle, DOI significantly increases the number of headshakes. None of the doses of the deuterated compound of Example 1 (A2) have any significant effect on this measure.

Acute oral administration of the deuterated compound of Example 1 (A2) (1, 5, and 10 mg/kg) shows no significant increase in the number of headshakes compared to vehicle. DOI (3 mg/kg) significantly increases headshake responses in the rats following acute i.p. injection.

### Example 10—DOI-Induced Headshake Response

Adult male Sprague Dawley rats are used. The deuterated compound of Example 1 (A2) (1, 5, and 10 mg/kg) is formulated in 0.5% methylcellulose solution and administered orally (PO) at a dose volume of 1 ml/kg 4 hours prior to test. DOI (3 mg/kg) is dissolved in saline and administered IP at a dose volume of 1 ml/kg (10 minutes prior to test). Ketanserin (1 mg/kg) is dissolved in saline and injected IP 30 minutes prior to DOI at a dose volume of 1 mg/kg.

Animals are administered vehicle, ketanserin, or test compound and returned to their holding cage for the appropriate pretreatment time (4 hours for the deuterated compound of Example 1 (A2) and 30 minutes for ketanserin). Rats are then injected with DOI and headshakes are recorded 10 minutes after DOI injection for 10 minutes using video cameras. The headshake response is a rapid, rhythmic shaking of the head in a radial motion. Data is analyzed by ANOVA followed by post hoc analysis where appropriate.

Dunnett's post analysis finds that, compared to vehicle, DOI significantly increases the number of headshakes. Ketanserin and the deuterated compound of Example 1 (A2) (1, 5, and 10 mg/kg) significantly attenuate DOI-induced headshake responses.

Acute oral administration of the deuterated compound of Example 1 (A2) (1, 5, and 10 mg/kg) decreases DOI-induced headshakes compared to vehicle. Ketanserin (1 mg/kg) also decreases the number of headshake responses induced by DOI following acute i.p. injection.

1. A compound of Formula I: 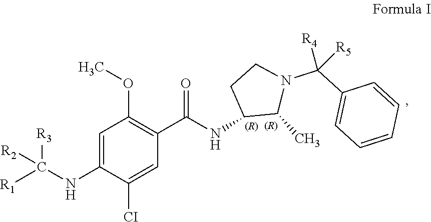
wherein:
R₁, R₂, R₃, R₄, and R₅ are independently selected from H and D; and
at least one of R₁, R₂, and R₃ is D;
in free or salt form.

2. The compound according to claim 1, wherein the compound is in free form.

3. The compound according to claim 1, wherein R₁, R₂, and R₃ are D.

4. (canceled)

5. A compound, wherein the compound is: 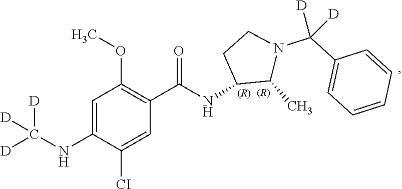
in free or pharmaceutically acceptable salt form.

6. The compound according to claim 1, wherein the compound, in free or pharmaceutically acceptable salt form, has greater than 90% incorporation of deuterium at one or more positions designated as deuterium.

7. A pharmaceutical composition, wherein the pharmaceutical composition comprises the compound according to claim 1, in free or pharmaceutically acceptable salt form, and a pharmaceutically acceptable carrier.

8. (canceled)

9. A method for treatment of an affective disorder or an anxiety disorder in a patient in need thereof, wherein the method comprises administering to the patient an effective amount of the compound according to claim 5, in free or pharmaceutically acceptable salt form.

10. A method for treatment of depression, an anxiety disorder, psychosis, schizophrenia, schizoaffective disorder, post-traumatic stress disorder (PTSD), attention-deficit/hyperactivity disorder (ADHD), Tourette syndrome, anorexia nervosa, bulimia nervosa, binge-eating disorder, body dysmorphic disorder, obsessive compulsive disorder, addiction, bipolar disorder, or a migraine in a patient in need thereof, wherein the method comprises administering to the patient an effective amount of the compound according to claim 5, in free or pharmaceutically acceptable salt form.

11. The method according to claim 10, wherein the anxiety disorder is panic disorder, social anxiety disorder, a phobia, or generalized anxiety disorder.

12. A method for treatment of anhedonia, depression associated with anhedonia, suicidal ideation, anxious depression, inflammatory depression, treatment-resistant depression, dysthymia, bipolar depression, psychotic depression, or post-psychotic depression in a patient in need thereof, wherein the method comprises administering to the patient an effective amount of the compound according to claim 5, in free or pharmaceutically acceptable salt form.

13. The method according to claim 12, wherein the method is for treatment of anxious depression.

14. The method according to claim 10, wherein the method is for treatment of melancholic depression.

15. A method for treatment of major depressive disorder in a patient in need thereof, wherein the method comprises administering to the patient an effective amount of the compound according to claim 5, in free or pharmaceutically acceptable salt form.

16. A method for treatment of a substance use disorder in a patient in need thereof, wherein the method comprises administering to the patient an effective amount of the compound according to claim 5, in free or pharmaceutically acceptable salt form.

17. (canceled)

18. (canceled)

19. (canceled)

20. The compound according to claim 5, wherein the compound is in free form.

21. The compound according to claim 5, wherein the compound, in free or pharmaceutically acceptable salt form, has greater than 90% incorporation of deuterium at one or more positions designated as deuterium.

22. The compound according to claim 20, wherein the compound, in free form, has greater than 90% incorporation of deuterium at one or more positions designated as deuterium.

23. A pharmaceutical composition, wherein the pharmaceutical composition comprises the compound according to claim 5, in free or pharmaceutically acceptable salt form, and a pharmaceutically acceptable carrier.

24. A pharmaceutical composition, wherein the pharmaceutical composition comprises the compound according to claim 20, in free form, and a pharmaceutically acceptable carrier.

25. A pharmaceutical composition, wherein the pharmaceutical composition comprises the compound according to claim 21, in free or pharmaceutically acceptable salt form, and a pharmaceutically acceptable carrier.

26. A pharmaceutical composition, wherein the pharmaceutical composition comprises the compound according to claim 22, in free form, and a pharmaceutically acceptable carrier.
